(19) United States
(12) Patent Application Publication
(10) Pub. No.: US 2025/0163472 A1
(43) Pub. Date: May 22, 2025

(54) Cell-free DNA for Use in Genotyping

(71) Applicant: ABS Global, Inc., DeForest, WI (US)

(72) Inventors: Sandeep RAJPUT, Sun Prairie, WI (US); Namdori MTANGO, Madison, WI (US); Rebecca Lynn KRISHER, Cottage Grove, WI (US)

(73) Assignee: ABS Global, Inc., DeForest, WI (US)

(21) Appl. No.: 18/729,004

(22) PCT Filed: Jan. 11, 2023
(86) PCT No.: PCT/US2023/060458

§ 371 Date: Jul. 15, 2024

Related U.S. Application Data
(60) Provisional application No. 63/299,739, filed on Jan. 14, 2022.
(60) Provisional application No. 63/269,926, filed on Mar. 25, 2022.

(51) Int. Cl. C12N 15/873; C12Q 1/6806; C12Q 1/6844; C12Q 1/6883

(52) U.S. Cl. CPC C12N 15/873; C12Q 1/6806; C12Q 1/6844; C12Q 1/6883; C12Q 2600/124

(57) ABSTRACT

The present invention provides rapid genomic diagnostic methods of in vitro produced embryos. Cell-free DNA can be obtained from the culture medium of blastocysts, and then added to a LAMP or PCR amplification reaction to detect the presence or absence of a certain DNA sequence. Colorimetric or fluorescent indicators such as metallochromatic dyes, intercalating dyes, or pH indicator dyes can be added to the reaction so that amplification can be tracked without additional steps. Provided are methods of sex determination and detection of a specific gene edit, as well adverse or desirable phenotypes. Methods of genotyping using cell-free DNA are also provided.

### CROSS REFERENCE TO RELATED APPLICATION

This application claims the benefit of and priority to U.S. Provisional Application 63/299,739, filed on Jan. 14, 2022. This application also claims the benefit of and priority to U.S. Provisional Application 63/269,926, filed on Mar. 25, 2022. 63/299,739 and 63/269,926 are each hereby incorporated by reference, each in their entirety.

### INCORPORATION OF SEQUENCE LISTING

The Sequence Listing, including the file named RB-34-2022-WO1-SEQLST.xml, which is 89,040 bytes in size, was created on Jan. 10, 2023 and is hereby incorporated by reference in its entirety.

### TECHNICAL FIELD

The present application relates to the field of in vitro produced embryos and livestock genetic analysis.

### BACKGROUND ART

Genetic analysis forms the basis for much of the current process of selection of parents for breeding the next generation of livestock. Narrow margins pressure farmers to keep costs low; the cost to raise an unwanted animal long enough to sell is nearly twice as much as disposing of it immediately—for example, in 2018, the Guardian reported that immediate disposal of a male calf in a dairy farm cost £9, but raising the calf long enough to sell it for beef production cost £30. Therefore, early genetic analysis can be key to farm efficiency.

Further, use of in vitro fertilization (IVF) to make in vitro produced (IVP) embryos has been increasing in various livestock industries, including the dairy industry. It is possible to make more in vitro produced embryos than there are available surrogate mothers at any given time. While several methods of cryogenic freezing are known in the art, better methods of selecting embryos for implantation in surrogate mothers are needed.

Previous methods of genomic screening or genotyping of bovine embryos include biopsy of the embryo, which can detract from the health of the embryo.

### SUMMARY

In general, the present teachings are useful for rapid, non-invasive methods of analyzing DNA of in vitro cultured embryos for specific traits, such as but without limitation, the sex of the embryo. Specifically, DNA accumulated in the medium can be amplified and then detected using various indicators.

In various embodiments, a method of making an animal or a cell line having a trait from an in vitro produced embryo can comprise: combining male and female gametes in vitro to form an embryo or producing a parthenogenetic embryo; culturing the embryo or parthenogenetic embryo for a period of time sufficient for cell-free DNA to accumulate in medium; adding the medium to an amplification reaction mix comprising primers targeted to a trait-related locus and at least one turbidimetric, colorimetric, or fluorescent indicator; amplifying the cell-free DNA; determining that the primers amplify the trait-related locus for the trait or determining that the primers do not amplify the trait-related locus for the trait based on a signal from the at least one turbidimetric, colorimetric, or fluorescent indicator; selecting the embryo or parthenogenetic embryo; and implanting the selected embryo into a surrogate mother or using cells from the selected embryo or selected parthenogenetic embryo to create a cell line. In various configurations, selecting the embryo or parthenogenetic embryo can comprise selecting the embryo or parthenogenetic embryo if the trait-related locus is amplified by the primers. In various configurations, selecting the embryo or parthenogenetic embryo can comprise selecting the embryo if the trait-related locus is not amplified by the primers. In various configurations, the amplifying the cell-free DNA can be by loop-mediated isothermal amplification (LAMP). In various configurations, the trait can be a health trait, a reproductive trait, a disease resistance trait, an anatomical trait, a desired gene edit, or sex of the embryo. In various configurations, the trait can be sex of the embryo and the locus can be on the Y chromosome. In various configurations, the trait-related locus can be TSPY or HSFY. In various configurations, the primers can comprise a set of oligonucleotides consisting of: SEQ ID NO: 1-6, SEQ ID NO: 7-12, SEQ ID NO: 3-6, and 13-14, SEQ ID NO: 9-10, 15-17, and 34, SEQ ID NO: 7-10, 17, and 34, SEQ ID NO: 9-12 and 15-16, SEQ ID NO: 26-31, SEQ ID NO: 28-33, SEQ ID NO: 5-6 and 13-14, SEQ ID NO: 15-17 and 34, SEQ ID NO: 26-27 and 30-31, SEQ ID NO: 1-2 and 5-6, SEQ ID NO: 7-8, 17, and 34, SEQ ID NO: 30-33, or SEQ ID NO: 44-49. In various configurations, the trait-related locus can be HSFY. In various configurations, the primers can comprise a set of oligonucleotides consisting of SEQ ID NO: 44-49. In various configurations, a female embryo can be desired and the embryo can be selected if the at least one turbidimetric, colorimetric, or fluorescent indicator indicates that the primers do not amplify TSPY or HSFY. In various configurations, a male embryo can be desired and the embryo can be selected if the at least one turbidimetric, colorimetric or fluorescent indicator indicates that the primers do amplify TSPY or HSFY.

In some embodiments, the present teachings can include a kit comprising: amplification reagents, a set of primers directed to a particular locus, and at least one turbidimetric, colorimetric, or fluorescent indicator. In various configurations, the amplification reagents can comprise Bst Polymerase and the set of primers comprises at least four primers directed to the locus. In various configurations, the kit can be for sex determination of an embryo, and the particular locus can be TSPY or HSFY.

In various configurations, the at least one turbidimetric, colorimetric, or fluorescent indicator in the method or kit of the present teachings can be a fluorescent indicator. In some configurations, the fluorescent indicator can be an intercalating indicator. In various configurations, the at least one turbidimetric, colorimetric, or fluorescent indicator in the method or kit of the present teachings can be selected from the group consisting of phenol red, bromothymol blue, thymol blue, bromocresol purple, thymolphthalein, phenolphthalein, neutral red, brilliant yellow, cresol red, 4-(2-pyridylazo) resorcinol (PAR), hydroxynaphthol blue, SYTO 9, SYTO 13, SYTO 16, SYTO 64, SYTO 82, Boxto, Miami Green, Miami Yellow, Miami Orange, YOPRO 1, SYTO 62, TOPRO 3, SYTO 60, EvaGreen, POPO 3, DCS1, SYBR Green I, BOBO 3, Pico 488, VIC1, and TOTO 3.

In various configurations, the at least one turbidimetric, colorimetric, or fluorescent indicator in the method or kit of the present teachings can comprise SYTO82. In various configurations, the amplification reagents in the method or kit of the present teachings can comprise Bst Polymerase, the set of primers can comprise a set of primers consisting of SEQ ID NO: 1-6 or SEQ ID NO: 46-49, and the at least one turbidimetric, colorimetric, or fluorescent indicator can comprise SYTO82.

In various embodiments, a method of selecting an in vitro produced embryo for implantation can comprise: combining male and female gametes in vitro to form an embryo; culturing the embryo for a period of time sufficient for cell-free DNA to accumulate in medium; adding the medium to a whole genome amplification reaction mix; amplifying the cell-free DNA using a whole genome amplification mix to obtain an amplified genome; determining the genotype of the embryo; and implanting the embryo.

In various embodiments, a method of making a female animal or a female cell line from an in vitro produced embryo can comprise: combining male and female gametes in vitro to form an embryo or producing a parthenogenetic embryo; culturing the embryo or parthenogenetic embryo for a period of time sufficient for cell-free DNA to accumulate in medium; adding the medium to a LAMP amplification reaction mix comprising a set of primers targeted to a TSPY locus comprising the nucleic acid sequences consisting of SEQ ID NO: 1-6, SEQ ID NO: 7-12, SEQ ID NO: 3-6, and 13-14, SEQ ID NO: 9-10, 15-17, and 34, SEQ ID NO: 7-10, 17, and 34, SEQ ID NO: 9-12 and 15-16, SEQ ID NO: 26-31, SEQ ID NO: 28-33, SEQ ID NO: 5-6 and 13-14, SEQ ID NO: 15-17 and 34, SEQ ID NO: 26-27 and 30-31, SEQ ID NO: 1-2 and 5-6, SEQ ID NO: 7-8, 17, and 34, or SEQ ID NO: 30-33 or a set of primers targeted to an HSFY locus comprising the nucleic acid sequences consisting of SEQ ID NO: 46-49 and a colorimetric indicator or a fluorescent indicator; amplifying the cell-free DNA; determining that the primers do not amplify the TSPY or HSFY locus based on a signal from the colorimetric indicator or fluorescent indicator; selecting the embryo or parthenogenetic embryo; and implanting the selected embryo into a surrogate mother or using cells from the embryo or parthenogenetic embryo to create a cell line.

In various embodiments, a method of making a male animal or a male cell line from an in vitro produced embryo can comprise: combining male and female gametes in vitro to form an embryo or producing a parthenogenetic embryo; culturing the embryo or parthenogenetic embryo for a period of time sufficient for cell-free DNA to accumulate in medium; adding the medium to a LAMP amplification reaction mix comprising a set of primers targeted to a TSPY locus comprising the nucleic acid sequences consisting of SEQ ID NO: 1-6, SEQ ID NO: 7-12, SEQ ID NO: 3-6, and 13-14, SEQ ID NO: 9-10, 15-17, and 34, SEQ ID NO: 7-10, 17, and 34, SEQ ID NO: 9-12 and 15-16, SEQ ID NO: 26-31, SEQ ID NO: 28-33, SEQ ID NO: 5-6 and 13-14, SEQ ID NO: 15-17 and 34, SEQ ID NO: 26-27 and 30-31, SEQ ID NO: 1-2 and 5-6, SEQ ID NO: 7-8, 17, and 34, or SEQ ID NO: 30-33, or a set of primers targeted to an HSFY locus comprising the nucleic acid sequences consisting of SEQ ID NO: 46-49 and a colorimetric indicator and a fluorescent indicator; amplifying the cell-free DNA; determining that the primers amplify the TSPY or HSFY locus based on a signal from the colorimetric indicator or the fluorescent indicator; selecting the embryo or parthenogenetic embryo; and implanting the selected embryo into a surrogate mother or using cells from the parthenogenetic embryo to create a cell line.

In various embodiments, a method of making an animal or a cell line having a desirable trait from an in vitro produced embryo can comprise: combining male and female gametes in vitro to form an embryo or producing a parthenogenetic embryo; culturing the embryo or parthenogenetic embryo for a period of time sufficient for cell-free DNA to accumulate in medium; adding the medium to an amplification reaction mix comprising primers targeted to a trait-related locus and at least one turbidimetric, colorimetric, or fluorescent indicator; amplifying the cell-free DNA; determining that the primers do not amplify a locus for an undesirable trait based on a signal from the at least one turbidimetric, colorimetric, or fluorescent indicator; selecting the embryo or parthenogenetic embryo; and implanting the selected embryo into a surrogate mother or using cells from the embryo or parthenogenetic embryo to create a cell line.

In various embodiments, a method of making an animal or a cell line having a desirable trait from an in vitro produced embryo can comprise: combining male and female gametes in vitro to form an embryo or producing a parthenogenetic embryo; culturing the embryo or parthenogenetic embryo for a period of time sufficient for cell-free DNA to accumulate in medium; adding the medium to an amplification reaction mix comprising primers targeted to a trait-related locus and at least one turbidimetric, colorimetric, or fluorescent indicator; amplifying the cell-free DNA; determining that the primers amplify a locus for the desirable trait; selecting the embryo or parthenogenetic embryo; and implanting the selected embryo into a surrogate mother or using cells from the embryo or parthenogenetic embryo to create a cell line.

In various embodiments, a method of selecting an in vitro produced embryo for creating a cell line can comprise: combining male and female gametes in vitro to form an embryo or producing a parthenogenetic embryo; culturing the embryo or parthenogenetic embryo for a period of time sufficient for cell-free DNA to accumulate in medium; adding the medium to a whole genome amplification reaction mix; amplifying the cell-free DNA to obtain an amplified genome; determining a genotype of the embryo or parthenogenetic embryo; and using cells from the embryo or the parthenogenetic embryo to create a cell line.

The present teachings also encompass an embryo or parthenogenetic embryo produced according to any method of the present teachings.

In various configurations, a method of the present teachings can further comprise determining a value based on a genotype-based value model and selecting the embryo or parthenogenetic embryo based on the value.

In various configurations, the whole genome amplification mix can be selected from the group consisting of a primer extension preamplification mix, a degenerate oligonucleotide primed-polymerase chain reaction mix, and a multiple displacement amplification mix. In various configurations, the whole genome amplification mix can be a primer extension preamplification mix. In various configurations, the whole genome amplification mix can be a degenerate oligonucleotide primed-polymerase chain reaction mix. In various configurations, the whole genome amplification mix can be a multiple displacement amplification mix.

In various configurations, the period of time is 3-9 days. In various configurations, the period of time can be about 7 days.

In various configurations, the method of the present teachings can further comprise determining a value based on a genotype-based value model and selecting the embryo or parthenogenetic embryo based on the value.

In various configurations, the trait-related locus for a method or kit of the present teachings can be a target of gene editing for a desired edit, and the primers are directed to the desired edit.

In various configurations, the embryo or parthenogenetic embryo suitable for use in the present teachings can be a non-human mammalian embryo or a non-human parthenogenetic mammalian embryo. In various configurations, the embryo or parthenogenetic embryo suitable for use in the present teachings can be a bovine embryo, a bovine parthenogenetic embryo, a porcine embryo, or a porcine parthenogenetic embryo. In various configurations, determining the genotype of the embryo or parthenogenetic embryo can comprise sequencing the amplified genome using a SNP array.

In various configurations, the embryo or parthenogenetic embryo used in the present teachings can be a non-human embryo or a non-human parthenogenetic embryo.

In some embodiments, a method of making an animal or a cell line having a desirable trait from an in vitro produced embryo can comprise combining male and female gametes in vitro to form an embryo. In various embodiments, the in vitro production of an embryo can comprise producing a parthenogenetic embryo; culturing the embryo for a period of time sufficient for cell-free DNA to accumulate in the medium; exposing medium from the embryo culture to an amplification reaction mix comprising primers targeted to a trait-related locus and at least one indicator; amplifying the cell-free DNA; detecting that the primers amplify a locus for the desirable trait or determining that the primers do not amplify a locus for an undesirable trait based on a signal from the at least one indicator; selecting the embryo; and implanting the selected embryo into a surrogate mother and gestating until birth or using cells from the embryo to create a cell line. In some configurations, the at least one indicator can be a colorimetric, fluorescent, or tubidimetric indicator. In some configurations, the at least one indicator can be a turbidimetric indicator. In some configurations, the at least one indicator can be a colorimetric indicator. In various configurations, the at least one indicator can be a fluorescent indicator. In various configurations, the amplifying the cell-free DNA can comprise loop-mediated isothermal amplification (LAMP). In some configurations, the period of time can be 3-9 days. In various configurations, the period of time can be 7 days.

In various configurations, the trait can be a health trait, a reproductive trait, a disease resistance trait, an anatomical trait, a desired gene edit, or sex of the embryo. In various configurations, the trait can be sex of the embryo and the locus can be on the Y chromosome. In some configurations, the locus can be TSPY. In some configurations, the primers can comprise a set of primers consisting of SEQ ID NO: 1-6, SEQ ID NO: 7-12, SEQ ID NO: 3-6, and 13-14, SEQ ID NO: 9-10, 15-17, and 34, SEQ ID NO: 7-10, 17, and 34, SEQ ID NO: 9-12, and 15-16, SEQ ID NO: 26-31, SEQ ID NO: 28-33, SEQ ID NO: 5-6 and 13-14, SEQ ID NO: 15-17 and 34, SEQ ID NO: 26-27 and 30-31, SEQ ID NO: 1-2, 5-6, SEQ ID NO: 7-8, 17, and 34, and SEQ ID NO: 30-33. In some configurations, the primers can be SEQ ID NO: 1-6. In some configurations, the primers can be SEQ ID NO: 7-12. In some configurations, the primers can be SEQ ID NO: 3-6 and 13-14. In some configurations, the primers can be SEQ ID NO: 9-10, 15-17, and 34. In some configurations, the primers can be SEQ ID NO: 7-10, 17, and 34. In some configurations, the primers can be SEQ ID NO: 9-12 and 15-16. In some configurations, the primers can be SEQ ID NO: 26-31. In some configurations, the primers can be SEQ ID NO: 28-33. In some configurations, the primers can be SEQ ID NO: 5-6 and 13-14. In some configurations, the primers can be SEQ ID NO: 15-17 and 34. In some configurations, the primers can be SEQ ID NO: 26-27 and 30-31. In some configurations, the primers can be SEQ ID NO: 1-2 and 5-6. In some configurations, the primers can be SEQ ID NO: 7-8, 17, and 34. In some configurations, the primers can be SEQ ID NO: 30-33. In various configurations, the embryo desired is female and the embryo can be selected if the at least one turbidimetric, colorimetric, or fluorescent indicator indicates that the primers do not amplify TSPY. In various configurations, the embryo desired is male and the embryo can be selected if the at least one indicator indicates that the primers do amplify TSPY.

In various configurations, the at least one indicator can be a pH indicator, an intercalating dye, or a metallochromic indicator. In various configurations, the at least one indicator can be phenol red, bromothymol blue, thymol blue, bromocresol purple, thymolphthalein, phenolphthalein, neutral red, brilliant yellow, cresol red, 4-(2-pyridylazo) resorcinol (PAR), or hydroxynaphthol blue. In various configurations, the at least one indicator can be phenol red.

In various configurations, a method of the present teachings can further comprise determining a value based on a genotype-based value model. In some configurations, the genotype-based value model can be a genomic estimated breeding value (GEBV), an estimated breeding value (EBV), or a best linear unbiased prediction (BLUP).

In various configurations, the locus can be a target of gene editing for a desired edit, and the primers can be directed to the desired edit.

In various embodiments, the present teachings provide for and include a kit comprising: amplification reagents, a set of primers directed to a particular locus, and at least one turbidimetric, colorimetric, or fluorescent indicator. In some configurations, the at least one turbidimetric, colorimetric, or fluorescent indicator can be a colorimetric, or fluorescent indicator. In some configurations, the at least one turbidimetric, colorimetric, or fluorescent indicator can be a turbidimetric indicator. In some configurations, the at least one turbidimetric, colorimetric, or fluorescent indicator can be a colorimetric indicator. In various configurations, the at least one turbidimetric, colorimetric, or fluorescent indicator can be a fluorescent indicator. In some configurations, the amplification reagents can comprise a DNA polymerase. In some configurations, the amplification reagents can comprise a nucleic acid polymerase selected from the group consisting of a TAQ polymerase, Bsu DNA polymerase, and Bst DNA Polymerase. In various configurations, the amplification reagents can comprise Bst Polymerase and the set of primers can comprise four primers directed to the locus. In various configurations, the amplification reagents can comprise Bst Polymerase and the set of primers can comprise six primers directed to the locus.

In various configurations, the at least one turbidimetric, colorimetric, or fluorescent indicator can be phenol red, bromothymol blue, thymol blue, bromocresol purple, thymolphthalein, phenolphthalein, neutral red, brilliant yellow, cresol red, 4-(2-pyridylazo) resorcinol (PAR), or hydroxynaphthol blue.

In various configurations, at least one the at least one turbidimetric, colorimetric, or fluorescent indicator can be phenol red. In various configurations, the kit can be for sex determination of an embryo, and the set of primers can be directed to TSPY. In various configurations, the amplification reagents can comprise Bst Polymerase, the set of primers can have sequences SEQ ID NO: 1-6, and the at least one turbidimetric, colorimetric, or fluorescent indicator molecule can be phenol red.

In various embodiments, the present teachings provide for and include a method of selecting an in vitro produced embryo for implantation comprising: combining male and female gametes in vitro to form an embryo or producing a parthenogenetic embryo; culturing the embryo for a period of time sufficient for cell-free DNA to accumulate in the medium; adding medium from the embryo culture to a whole genome amplification reaction mix; amplifying the cell-free DNA to obtain an amplified genome; determining the genotype of the embryo; and implanting the embryo. In some configurations, the embryo can be frozen prior to implantation. In some configurations, the embryo can be slow frozen. In various configurations, the embryo can be vitrified. In various configurations, the method can further comprise determining a value based on a genotype-based value model. In some configurations the genotype-based value model can be an estimated breeding value (EBV), and wherein the selecting the embryo based on the genotype comprises selecting the embryo based on the EBV. In various configurations, the whole genome amplification mix can be primer extension preamplification mix, degenerate oligonucleotide primed-polymerase chain reaction mix, multiple displacement amplification mix, or OmniPlex whole genome amplification mix. In various configurations, the embryo can be a non-human mammalian embryo. In various configurations, the embryo can be a bovine embryo or a porcine embryo. In various configurations, the embryo can be a Bos taurus, Bos indicus, Sus scrofa, or a Bubalus bubalis embryo. In some configurations, the determining the genotype of the embryo can comprise genotyping the amplified genome using a SNP array.

In various embodiments, the present teachings provide for and include a kit comprising: a whole genome amplification reaction mix and a SNP array. In some configurations, the whole genome amplification mix can be selected from the group consisting of primer extension preamplification mix, degenerate oligonucleotide primed-polymerase chain reaction mix, multiple displacement amplification mix, and OmniPlex whole genome amplification mix.

In various embodiments, the present teachings provide for and include method of making a female animal or a female cell line from an in vitro produced embryo comprising: combining male and female gametes in vitro to form an embryo or producing a parthenogenetic embryo; culturing the embryo for a period of time sufficient for cell-free DNA to accumulate in the medium; adding medium from the embryo culture to a LAMP amplification reaction mix comprising a set of primers targeted to a TSPY locus comprising the nucleic acid sequences consisting of SEQ ID NO: 1-6, SEQ ID NO: 7-12, SEQ ID NO: 3-6, and 13-14 SEQ ID NO: 9-10, 15-17, and 34, SEQ ID NO: 7-10, 17, and 34, SEQ ID NO: 9-12, and 15-16, SEQ ID NO: 26-31, SEQ ID NO: 28-33, SEQ ID NO: 5-6 and 13-14, SEQ ID NO: 15-17 and 34, SEQ ID NO: 26-27 and 30-31, SEQ ID NO: 1-2 and 5-6, SEQ ID NO: 7-8, 17, and 34, and SEQ ID NO: 30-33 and a colorimetric indicator; amplifying the cell-free DNA; determining that the primers do not amplify the TSPY locus based on a signal from the colorimetric indicator; selecting the embryo; and implanting the selected embryo into a surrogate mother and gestating until birth or using cells from the embryo to create a cell line. In some configurations, the period of time can be 3-9 days. In some configurations, the period of time can be 7 days.

In various embodiments, the present teachings provide for and include a method of making a male animal or a male cell line from an in vitro produced embryo comprising: combining male and female gametes in vitro to form an embryo or producing a parthenogenetic embryo; culturing the embryo for a period of time sufficient for cell-free DNA to accumulate in the medium; adding medium from the embryo culture to a LAMP amplification reaction mix comprising a set of primers targeted to a TSPY locus comprising the nucleic acid sequences consisting of SEQ ID NO: 1-6, SEQ ID NO: 7-12, SEQ ID NO: 3-6, and 13-14, SEQ ID NO: 9-10, 15-17, and 34, SEQ ID NO: 7-10, 17, and 34, SEQ ID NO: 9-12 and 15-16, SEQ ID NO: 26-31, SEQ ID NO: 28-33, SEQ ID NO: 5-6 and 13-14, SEQ ID NO: 15-17 and 34, SEQ ID NO: 26-27 and 30-31, SEQ ID NO: 1-2 and 5-6, SEQ ID NO: 7-8, 17, and 34, and SEQ ID NO: 30-33, and a colorimetric indicator; amplifying the cell-free DNA; determining that the primers amplify the TSPY locus based on a signal from the colorimetric indicator; selecting the embryo; and implanting the selected embryo into a surrogate mother and gestating until birth or using cells from the embryo to create a cell line. In some configurations, the period of time can be 3-9 days. In some configurations, the period of time can be 7 days.

An embryo having at least one desirable trait, the embryo produced by a method of the present teachings.

### DETAILED DESCRIPTION

The present teachings provide for and include non-invasive, rapid methods of screening in vitro produced embryos for a variety of traits, but especially for the sex of the embryo. The methods of the present teachings allow for amplification of cell-free DNA in cell culture media and then the use of at least one indicator to detect the presence of amplified DNA. The indicator can be a colorimetric, turbidimetric, or fluorescent indicator that changes appearance with or without stimulation to indicate the presence of amplification. Cell-free DNA is released into cell culture media during the culture period. Media is typically changed after three days of cell culture, and then the embryo is implanted after 7-9 days of culture. Cell-free DNA can be obtained from the conditioned medium from a few hours after the inception of the culture until the embryo is implanted in an animal, dissected to be transformed into a cell line, or used for other experimental purposes. Medium can be used for the amplification of the present teachings for up to 9 days in embryo culture. The medium removed at 3 days can be used as a source for cell-free DNA assays. Medium from day 4 to day 9 cultures can be used as a source of cell-free DNA. Medium from day 5 to day 9 cultures can be used as a source of cell-free DNA. Medium from day 5 to day 7 cultures can be used as a source of cell-free DNA. Medium from day 7 cultures can be used as a source of cell-free DNA.

The present teachings are suitable for any embryo that has an outer membrane (or shell) that is permeable or selectively permeable membrane to DNA, wherein the embryo requires growth in a liquid medium. The liquid medium can either be a specialized growth medium or water. Any specialized growth medium known to be suitable for the embryos in a particular application can be used. Serum free medium is advantageous as animal serum can contain DNA that may occasionally result in non-specific amplification products. These non-specific products are highly primer dependent and can vary from application to application. Further, some growth mediums have buffer effects, and larger volumes of growth medium in a reaction may inhibit a pH change, which is one of several types of indicators that can be used in the present teachings. The present inventors have found that for IVC2, 1-3 μl of spent culture medium produces a detectable color change, but 4 μl or higher can inhibit the pH color change. For cultures with smaller DNA concentrations, other methods such as the use of intercalating dyes may have advantages over a pH indicator molecule. The present teachings can be used in any in vitro grown organism where DNA accumulates in the medium. Organisms that are known to produce cell-free DNA include humans and cattle. It is expected that the present teachings are especially useful for both terrestrial and aquatic livestock.

As used herein, “livestock” refers to animals that are farmed either on land (“terrestrial livestock”) or in aquaculture (“aquatic livestock”) for production of food for humans or other animals. Terrestrial livestock may include cattle, swine, goats, or sheep. Aquatic livestock may include fish or edible crustaceans such as shrimp.

In general, the present teachings are especially useful for characterizing embryos that have been created using IVF or parthenogenetic techniques. The present teachings are especially useful for selecting mammalian embryos for implantation into a surrogate mother. In some configurations, the embryo can be a cattle embryo, a bison embryo, a buffalo embryo, a swine embryo, a sheep embryo, or a goat embryo. In some configurations, the embryo can be a Bos taurus embryo, a Bos indicus embryo, a Bubalus bubalis embryo, or a Sus scrofa embryo. In various configurations, the embryo can be a salmon egg, a catfish egg, a tilapia egg, a trout egg, a striped bass egg, or a shrimp egg. The most commonly farmed species of shrimp belong to the Penaeidae family and include Pacific white shrimp (Penaeus vannamei), giant tiger prawn (P. monodon), Indian white shrimp (P. indicus), Kuruma shrimp (P. japonicus), and the giant freshwater prawn (Macrobrachium rosenbergii).

As herein, “bovine” refers to animals that are members of the subfamily Bovinae which includes (but is not limited to) the genera Bison (Bison), Bos (Cattle), and Bubalis (buffalo). Bovine species include Bos taurus, Bos indicus, and Bubalus bubalis.

As used herein, “porcine” refers to a variety of taxa of animals commonly referred to as varieties of “pig”—especially the subfamily Suidae, which includes both wild and domestic pigs, especially of the genus Sus, which includes Sus scrofa and Sus scrofa domesticus.

As used herein, “cell-free DNA” refers to embryonic DNA that accumulates in the medium of an embryo culture and that is accessible without biopsying the embryo. Preferably, cell-free DNA is obtained without the need to manipulate or disrupt embryonic cells to obtain the DNA. Skilled artisans will recognize that the medium may still contain cellular debris that has been naturally shed into the culture, and that samples of cell-free DNA may include such cellular debris. The cell-free DNA may be from many different sources, including micro or macro vesicles released from the embryo during in vitro culture.

Alternatively or in addition, the methods described herein may be performed on other biofluids that can contain extracellular DNA, such as saliva, milk, urine, or semen.

As used herein, a “colorimetric or fluorescent indicator” is a molecule that changes signal in response to a chemical change in the reaction mix when amplification occurs. A “chemical change” can include a change in pH, an increase in DNA concentration, or other measurable changes in the reaction mix. “Signal” in this instance means a change in visual emissions—either color changes that can be seen visually or fluorescence changes that can be detected with proper excitation of the molecule. Alternatively, signal may refer to an observed change in turbidity, which could also indicate that an amplification reaction has occurred.

As used herein, the term “dNTPs” refers to a mix of deoxyriboguansine triphosphate, deoxyribocytosine triphosphate, deoxyriboadenosine triphosphate, and deoxyribothymine triphosphate.

In general, in vitro production of mammalian livestock embryos is a three-step process involving oocyte maturation, oocyte fertilization and in vitro culture. Only 30-40% of such oocytes reach the blastocyst stage, at which point they can be transferred to a recipient or frozen for future use. At any stage in the culture process, the spent media, which was previously discarded, can be used to screen the genotype of the embryo by amplifying cell-free DNA. The quality of the oocyte can dramatically impact the proportion of immature oocytes that form blastocysts; the post-fertilization culture environment has a major influence on the quality of the blastocyst. In some embodiments, use of sperm bearing a specific sex chromosome in conjunction with in vitro embryo production is a potentially efficient means of obtaining offspring of the desired sex. Concerns regarding the use of sexed semen technology include the apparent lower fertility of sorted sperm, the lower survival of sorted sperm after cryopreservation and the reduced number of sperm that could be separated in a specified time period. Assessment of embryo quality is also a challenge. Morphological assessment is at present the most popular method for embryo selection prior to transfer. Other non-invasive assessment methods include the timing of the first cleavage division which has been linked to developmental ability. Quantitative examination of gene expression is an additional valuable tool to assess the viability of cultured embryos. However, such quantitative measurements require invasive protocols such as embryo biopsy. Qualitative assays of the present teachings may also be used to assess gene expression and quality of the embryo. For example, genotyping can be used to screen traits related to embryo quality, and epigenetic genotyping or profiling could be used for viability markers using cell-free DNA instead of embryo biopsy. A substantial amount of evidence exists to demonstrate that the culture conditions to which the embryo is exposed, particularly in the post-fertilization period, can have perturbing effects on the partem of gene expression in the embryo with potentially important long-term consequences.

IVF is a technique in which oocytes are fertilized in vitro. In an exemplary IVF procedure, oocytes are extracted from a donor animal by a method of aspiration from the reproductive tract. Selected oocytes are then incubated for a period of about 24 hours; this is called the maturation period. After maturation, the eggs are fertilized about 18 to 22 hours after the co-culture has been made. The embryos stay in the medium until around the seventh day, when they are ready to be transferred. This technique has three main advantages over conventional in vivo embryo collection. With IVF, it is not necessary to superovulate the animals, nor is it necessary to synchronize them. This is a major breakthrough since the donor animals are not exposed to hormones that might compromise the reproductive soundness of the animals, and they can be worked without prior preparation time for the procedure. In some production systems, IVP efficiency can average about 30% of the oocytes harvested, although this quantity varies depending on the species, the breed, the donor animal, and also the mating. Further, animals can be aspirated every 14 days instead of every 60 days as in in vivo embryo collection. Finally, the animals can be harvested at a very young age, significantly increasing genetic improvement rates by reducing the generation interval for the animals with specific desirable traits.

Embryo transfer (ET) technology allows producers to obtain multiple progenies at once from genetically superior females. Fertilized embryos can be recovered from females (also called embryo donors) of superior genetic merit by surgical or nonsurgical techniques. Alternately, oocytes can be harvested and then fertilized in vitro. The genetically superior embryos are then transferred to females (also called embryo recipients or surrogate mothers) of lesser genetic merit. In cattle, efficient techniques can recover fertilized embryos without surgery, but only one or sometimes two embryos are typically produced during each normal reproductive cycle. To increase the number of embryos that can be recovered from genetically superior females, the embryo donor is treated with a hormone regimen to induce multiple ovulations, or superovulation.

Alternatively, or in addition, methods of the present teachings may be used with parthenogenetic embryos, which are oocytes that have been activated to undergo development in the absence of fertilization. Parthenogenetic embryos can be haploid or diploid, depending on whether or not the extrusion of the second polar body has been inhibited. Oocyte activation can be achieved by any method known in the art, including mechanical activation, electrical activation, and chemical activation. Chemical activation can be ionomycin activation, ethanol activation, hyaluronidase activation, Ca⁺² ionophores or chelators, cycloheximide, 6-dimethylaminopurine (6-DMAP), or inhibitors of protein synthesis. Methods of generating parthenogenetic embryos are discussed in U.S. Pat. No. 5,843,754, 10,190,093, Tiziana A. L., et al., BIOLOGY OF REPRODUCTION, 72, 2005, 1218-1223, and Kharche, S. D. and Birade, H. S., Advances in Bioscience and Biotechnology, 2013, 4, Article ID:28406.

Previous attempts at early embryonic genotyping have included various techniques of amplifying DNA extracted from cells biopsied from an embryo. Hirayama et al. (Theriogenology, 2004, 62, 887-896), for example, determined the sex of biopsied embryos using a presence/absence assay for S4, a male-specific sequence.

In contrast, the present invention uses a non-invasive technique for embryonic genotyping embryos produced by IVF without requiring embryo biopsy, cell culture, or cellular extractions. Instead, methods and products of the invention may be used to amplify and genetically or epigenetically analyze cell-free embryonic DNA in the fluid IVF medium, and to make breeding selections and to breed animals based on those selections. In general, the amplification step can be loop-mediated isothermal amplification (LAMP), polymerase chain reaction (PCR), or any other suitable polynucleotide amplification method known to those in the art. Alternatively, or in addition, the cell-free DNA may be used for embryonic genotyping or epigenotyping by performing whole genome amplification followed by genotyping or next generation sequencing. There are several methods of whole genome amplification known in the art, for example, primer extension preamplification, degenerate oligonucleotide primed-polymerase chain reaction, multiple displacement amplification, and OmniPlex whole genome amplification (see e.g., Zheng, Y-m, et al., J. Zhejiang Univ. Sci. B., 2011, 12, 1-11). There are also many commercial kits available from various scientific suppliers including New England Biolabs, Sigma-Aldrich (also known as Millipore Sigma), ILLUMINA®, and QIAGEN® (QIAGEN® Group, Hilden, Germany).

As used herein, “genotyping” or “genotype” refer, respectively, to any method of determining information about a genome or the results of any of those methods. Genotyping may refer to determining the allele of a single gene or locus, such as a haplotype, or identifying multiple genes, loci or SNPs. This can include a small number of loci, such as 1-50 or a large number of loci, such as on a microarray (such as 50 k or 100 k microarray chips), or anywhere in between. Small scale genotyping (e.g., about 1-100 loci) can be performed using a LAMP assay in tubes or on a 96 well plate with a plurality of primer sets (but only one primer set per well). Large scale genotyping can be performed using any of several commercial genotyping kits, such as, but without limitation, Thermofisher AXIOM® Trout Genotyping Array (Waltham, MA), GGP Shrimp 50K (Neogen), GGP Porcine 50 k kit (ILLUMINA®, Inc., San Diego, CA), PorcineSNP60 (ILLUMINA®), GGP Porcine LD Array (ILLUMINA®), AXIOM® Porcine Genotyping Array (Thermofisher Scientific), AXIOM® Porcine Breeders Array (Thermofisher), the BovineSNP50 DNA Analysis BeadChip (ILLUMINA®), BovineLD v2.0 Genotyping BeadChip (ILLUMINA®), GGP Bovine uLD chip (Neogen, Lansing, MI), Thermo Scientific Bovine Genotyping Kit (Thermofisher), the AXIOM® Bovine 100 k array, AXIOM® Buffalo Genotyping Array, Aquaculture, AXIOM® Salmon Genotyping Array (Salmo salar), AXIOM® Catfish Genotyping Array, AXIOM® Coho Salmon Genotyping Array, or the AXIOM® Trout Genotyping Array. Both ILLUMINA® and ThermoFisher offer custom, scalable SNP arrays of variable density that are also suitable for the present teachings. Low density microarrays and amplification-based multiplexed assays are especially useful with the present teachings when they use fewer markers and require relatively smaller amounts of DNA. The number of loci or SNPs that can be interrogated will depend on the quantity and identity of the DNA produced by a given type of embryo under given culture conditions. Skilled artisans will recognize that other genotyping kits or methods may be used to obtain like results, and will be able to choose a suitable array or other kit based on the application.

For single gene applications, some embodiments of the present teachings use LAMP-based assays to provide sequence-specific amplification. These embodiments use a strand-displacing DNA polymerase for isothermal nucleic acid amplification, and therefore do not require thermocycling, as is required for PCR (Gill, P. and Ghaemi, A., Nucleosides, Nucleotides, and Nucleic Acids, 2008, 27, 224-243; Kim, J. and Easley, C. J., Bioanalysis, 2011, 3, 227-239). In addition to the DNA polymerase, LAMP uses 4 core primers (forward inner primer (FIP), backward inner primer (BIP), forward outer primer (F3), and backward outer primer (B3)) recognizing 6 distinct sequence regions on the target, with two optional “loop” primers (F1C, B1C) containing sequence that results in loop structures which facilitate exponential amplification (Notomi, T., et al., Nucleic Acids Res., 2000, 28, E63). These loop primers can be added to increase reaction speed, resulting in 6 total primers used per target sequence (Nagamine, K., et al., 2002, Mol. Cel. Probes, 16, 223-229). Alternatively, the LAMP assay can be done with the inner primers and loop primers using a slightly longer incubation time with no loss in efficiency. The use of multiple target sequence regions confers a high degree of specificity to the reaction. The LAMP reaction rapidly generates amplification products as multimers of the target region in various sizes, and is substantial in total DNA synthesis (>10 μg, >50×PCR yield) (Notomi, T., et al., Nucleic Acids Res 2000, 28, e63; Nagamine, et al., Clin. Chem., 2001, 47, 1742-1743). LAMP is especially advantageous for use in the present teachings, as the amplification reaction occurs at a constant temperature (as opposed to PCR, which requires thermocycling). Further, LAMP provides the flexibility of using different indicators such as colorimetric, turbidity, and/or fluorescent indicators, which allow for detection of amplification of the target sequence without specialized equipment. Further, in larger scale applications, there are relatively inexpensive detectors for isothermal amplification reactions that can accommodate a strip of 8 PCR tubes at once—for example, but without limitation, the labgene-8C or labgene-8C2 (Labgene Scientific, Chatel-Saint-Denis, Switzerland).

In general, LAMP reactions suitable for use with the present teachings include: a DNA polymerase suitable for isothermal amplification of DNA; dNTPs (dATP, dGTP, dCTP, and dTTP); a set of four or six oligonucleotide primers or LNA enhanced primers, and at least one indicator that provides a signal in response to chemical changes in the reaction mix that occur during DNA amplification. In some embodiments, the indicator can be a colorimetric or fluorescent indicator that changes color or produces fluorescence, respectively, in response to chemical changes in the reaction mix that occur during DNA amplification. Alternatively, or in addition, colorimetric endpoint analysis can be combined with fluorescent DNA intercalating dyes for real-time detection of LAMP amplicons. Adding real-time fluorescence detection to a colorimetric LAMP assay can be beneficial during optimization and reaction kinetic studies. The simultaneous use of color indicators and fluorescent dyes may decrease fluorescence signal while not affecting real-time fluorescence detection time.

The DNA polymerase can be any polymerase suitable for use in an isothermal amplification reaction (e.g., in a LAMP reaction). Suitable DNA polymerases are known in the art and include strand displacing DNA polymerases. Strand displacing DNA polymerases include mesophilic DNA polymerases such as Bst polymerase or variants thereof and Bsu DNA polymerase or variants thereof. Various suitable Bst and Bsu polymerases are available from commercial vendors such as New England Biolabs. Other suitable polymerases are sold under the tradename Bolt polymerase by Varigen.

LAMP amplification is usually carried out at a constant temperature for 15 minutes to 2 hours—with both variables dependent on the primers and template involved. The LAMP amplification can be for 15, 20, 25, 30, 35, 40, 45, 50, or 55 minutes, or 1 hour, 1.25 hours, 1.5 hours, 1.75 hours, or 2 hours.

PCR has been known in the art for more than twenty years—the basic requirements are a heat stable DNA polymerase such as TAQ DNA polymerase, dNTPs, a set of two primers, and an amplification indicator for use in the present teachings. Optional magnesium cofactor sources, such as magnesium chloride, may also be included. Many PCR kits are known in the art, and available from various manufacturers such as, but without limitation, Promega, Roche, Thermofisher, Enzo Lifesciences, and New England Biolabs. PCR amplification requires a thermocycler that can cycle through denaturing, annealing, and extension temperatures. Reactions can take 45 minutes to several hours, depending on the length of the template and the speed of the heat stable polymerase.

In some embodiments, the amplification indicator can be a pH indicator. A pH indicator can indicate pH via color changes detectable in visible light, or can indicate pH via a change in fluorescence, so long as the molecule provides a change in signal (e.g., a change in color, or a change in color intensity or fluorescence intensity) in response to the pH changes induced by an amplification reaction. The DNA amplification reaction causes the pH of the reaction mix to change, leading the spectral or fluorescent properties of the indicator to change (e.g., the indicator changes color), which provides confirmation that amplification has occurred. During nucleic acid amplification, hydrogen ions accumulate in the reaction mixture so that the mixture becomes increasingly acidic with increasing amplification. pH indicator dyes change their color, color intensity, or fluorescent intensity, in response to the change in pH in the reaction mixture. Examples of pH indicator dyes include colorimetric dyes such as phenol red, cresol red, m-cresol purple, bromocresol purple, neutral red, phenolphthalein, naphtholphthaein, bromothymol blue, thymol blue, thymolphthalein, and brilliant yellow. There are also pH sensitive fluorescent dyes such as 2′,7′-Bis-(2-Carboxyethyl)-5-(and-6)-Carboxyfluorescein or a carboxyl seminaphthorhodafluor (e.g., SNARF-1). In certain embodiments, the colorimetric pH indicator is phenol red.

Alternatively, or in addition, the at least one indicator can comprise an indicator that is not pH sensitive, such as a metallochromic indicator dye. When the metallochromic indicator dye is used in an amplification reaction, the reaction alters the availability of one or more metal ions in the reaction mix, causing the spectral or fluorescent properties of the indicator to change (e.g., the indicator changes color), which provides confirmation that amplification has occurred. The metallochromic indicator dye can be 4-(2-pyridylazo) resorcinol (PAR). If PAR is used, the amplification reaction may additionally comprise manganese ions such that the PAR is complexed with Mn ions to form a PAR-Mn complex. DNA amplification in typical reactions will cause the dye to change from orange to yellow. Without being limited by theory, in the initial reaction mix, PAR is complexed with a metal ion such as manganese, which causes it to be orange. As amplification occurs, pyrophosphate accumulates in the reaction vessel and sequesters the metal ions with a higher affinity than PAR. This high affinity results in the dissociation of PAR from the metal ion, thus turning the PAR yellow. Another example of a metallochromic dye is hydroxynaphthol blue (Wastling, S. L., et al., PLoS, Negl. Trop. Dis., 2010, 4, e865).

The at least one indicator may also be an intercalating fluorescent dye such as, but without limitation, Pico Green, SYBR Safe, SYBR Green, Calcein, SYTO 9, SYTO 80, SYTO 81, SYTO 82, SYTO 83, SYTO 84, SYTO 85, SYTOX Orange, SYTO 9, SYTO 13, SYTO 16, SYTO 24, SYTO 60, SYTO 62, SYTO 64, SYTO 82, SYBR Green I, SYBR Gold, YOPRO1, TOTO1, TOTO3, BOBO3, POPO3, TOPRO3 (Invitrogen, United States), Eva Green (Biotium, United States), Boxto (TATA Biocentre, Sweden), Miami Green, Miami Yellow, Miami Orange (Kerafast, United States), Pico 488 (Lumiprobe, Germany), VIC, or Nuclear Green DCS1 (Abcam, United Kingdom). Preferred fluorescent dyes include SYTO 9, SYTO 13, SYTO 16, SYTO 64, SYTO 82, Boxto, Miami Green, Miami Yellow, Miami Orange, YOPRO 1, SYTO 62, TOPRO 3, SYTO 60, EvaGreen, POPO 3, DCS1, SYBR Green I, BOBO 3, Pico 488, and TOTO 3. Many suitable intercalating dyes are commercially available. Without being limited by theory, dsDNA-binding dye intercalates nonspecifically into dsDNA, allowing measurement of the amount of amplification product generated by LAMP or PCR. For example, SYBR Green fluorescence increases up to 1,000-fold upon intercalation with dsDNA.

Alternatively, or in addition, a combination of an intercalating dye and a pH sensitive dye can be used.

Alternatively, or in addition, the at least one indicator can be a set of oligonucleotide probes: one highly specific to the desired SNP and a second competitive “sink” probe that is complementary to the off-target version of the SNP. The SNP probe can be labeled with a fluorescent moiety and a quencher. The quencher can be removed as the probe is incorporated into the product, causing the probe to fluoresce. The presence of fluorescent signal thus indicating the presence of the SNP. The non-fluorescent sink probe can bind to the off-target SNP, preventing the fluorescent probe from binding to off-target forms. In various configurations, the on-target probe and the sink probe can be labeled in different colors. Variations on this technology are being developed in the art (Hyman, L. B., et al., bioRxiv preprint 2021 doi 0.1101/2021.03.29.437576; Li, Q., et al., Nucleic Acids Research, 2002, 30, e5) and are reviewed in Varona, M. and Anderson, J. L., ACS Omega 2021, 6, 3463-3469.

Other approaches for SNP detection include placing the primer at the location of the SNP which results in much faster amplification of the desired SNP over the non-desired SNP, using a peptide-nucleic acid and a locked nucleic acid to differentiate the two versions of the SNP, or multiple enzyme mediated reactions (Varona, M. and Anderson, J. L., ACS Omega 2021, 6, 3463-3469).

In some embodiments, in order to efficiently perform many reactions at once, the components of a LAMP reaction may be provided in a master mix or combined into a master mix during reaction set-up. In general, a master mix would include a DNA polymerase suitable for isothermal amplification of DNA, dNTPs (dATP, dGTP, dCTP, and dTTP), and an indicator. A master mix for amplifying many templates with the same primers can also comprise the primers for the desired reaction. The master mix can then be distributed to different tubes, and separate cell-free DNA templates (e.g., from spent growth medium from different embryos in individual culture) can then be added to each tube. (If not in the master mix, the sets of primers may also be added to each tube or well.)

The master mix may be dried or may be in a weakly buffered solution. In many embodiments, the master mix is a Loop-Mediated Isothermal Amplification (LAMP) master mix. However, the parameters tested herein may be applicable to master mixes for non-LAMP isothermal amplification reactions. Further, the components may be provided separately and mixed during execution of the method.

In some embodiments, the present teachings provide for and include a kit comprising the components of the amplification reaction. In embodiments where the master mix is dried, the master mix may be freeze dried, air dried, or lyophilized. The master mix may be immobilized, for example on paper, or on a natural or synthetic polymer. The dried master mix is reconstituted prior to use in an amplification reaction.

In embodiments where the master mix is in solution (e.g., following reconstitution), the master mix can be in a weakly buffered solution, such as in a Tris buffer. The weakly buffered solution preferably has a concentration less than 5 mM, such as less than 5 mM Tris or equivalent buffer. In one embodiment, the weakly buffered solution is in the range of 0.5 mM to 5 mM, such as 0.5 mM to 5 mM Tris or equivalent buffer. The pH of the master mix may be buffered in the range of pH 7.5-pH 9.0; such as in the range of pH 7.8-pH 8.5, or pH 8.1-pH 8.5. The liquid form of the master mix may be in any suitable reaction container.

The design of primers for PCR or LAMP is known in the art, and primers can be designed against any locus of a desired trait.

Exemplary LAMP primers for are provided in Table 1. B1.75 is bovine 1.715 satellite DNA (Genbank Accession No. V00125.1) repetitive sequence and can be used as a positive control to verify the presence of bovine DNA in cell culture medium. Testis specific protein Y (TSPY) genes are located on the Y chromosome and have a high copy number, making them excellent loci for determining the presence or absence of the Y chromosome, and therefore the sex of the embryo. Heat Shock Transcription Factor-Y linked (HSFY) is a housekeeping gene on the Y chromosome. Exemplary sets of primers for amplifying loci on the TSPY gene, HSFY gene, and B 1.75 gene are listed below in Table 1 and are listed by locus. Skilled artisans will recognize that although some primers can be reused, each set must be designed to work together, and reuse of a particular primer requires checking in silico that it is suitable to use with a different primer set.

TABLE 1
Exemplary LAMP Primers

| | SEQ | | |
|---|---|---|---|
| | ID | SEQ | |
| Locus | NO: | Name | Sequence |
| TSPY | 1 | TSPYN1 | TGACGCTTCCTCCTCTGATGGTTTTT |
| (set 1) | | FIPe | GAGCAAAACCGCAGGGCTTA |
| | 2 | TSPYN1 | CGCCATCATCCAGGGCATCCTTTTAT |
| | | BIPe | CAGGACGGAGACTTGAGG |
| | 3 | TSPYN1 | CCTGCAGGTAGAACTGAGCT |
| | | F3 | |
| | 4 | TSPYN1 | AGTCTTGATCTTGGTCGCTG |
| | | B3 | |
| | 5 | TSPYN1 | TCTTGCGCATGAACTGAACG |
| | | LF | |
| | 6 | TSPYN1 | CTGGCTTCTGGGCCAAAG |
| | | LB | |
| TSPY (2) | 7 | bTSPY1- | TGACGCTTCCTCCTCTGATGGTTTTT |
| | | FIBe | AGCTCTGAGCGTGAGCAA |
| | 8 | bTSPY1- | CGCCATCATCCAGGGCATCCTTTTGC |
| | | BIPe | TGATCAGGACGGAGACT |
| | 9 | bTSPY1 | CGCCCTGCAGGTAGAACTG |
| | | F3 | |
| | 10 | bTSPY1 | AGTCCATCATGTAGCCGAGA |
| | | B3 | |
| | 11 | bTSPY1- | TGAACTGAACGTAAGCCCT |
| | | LF | |
| | 12 | bTSPY1- | GGGCCAAAGCTATTATGAGCCA |
| | | LB | |
| TSPY (3) | 13 | TSPYN1 | TGACGCTTCCTCCTCTGATGGTGAGC |
| | | FIP | AAAACCGCAGGGCTTA |
| | 14 | TSPYN1 | CGCCATCATCCAGGGCATCCATCAGG |
| | | BIP | ACGGAGACTTGAGG |
| | 3 | TSPYN1 | CCTGCAGGTAGAACTGAGCT |
| | | F3 | |
| | 4 | TSPYN1 | AGTCTTGATCTTGGTCGCTG |
| | | B3 | |
| | 5 | TSPYN1 | TCTTGCGCATGAACTGAACG |
| | | LF | |
| | 6 | TSPYN1 | CTGGCTTCTGGGCCAAAG |
| | | LB | |
| TSPY (4) | 15 | TSPYN2 | TGACGCTTCCTCCTCTGATGGTAGCT |
| | | FIP | CTGAGCGTGAGCAA |
| | 16 | TSPYN2 | CGCCATCATCCAGGGCATCCGCTGAT |
| | | BIP | CAGGACGGAGACT |
| | 9 | TSPYN2 | CGCCCTGCAGGTAGAACTG |
| | | F3 | |
| | 10 | TSPYN2 | AGTCCATCATGTAGCCGAGA |
| | | B3 | |
| | 17 | TSPYN2 | CATGAACTGAACGTAAGCCCT |
| | | LF | |
| | 34 | TSPYN2 | GGCCAAAGCTATTATGAGCCA |
| | | LB | |
| TSPY (5) | 7 | TSPYN2 | TGACGCTTCCTCCTCTGATGGTTTTT |
| | | FIPe | AGCTCTGAGCGTGAGCAA |
| | 8 | TSPYN2 | CGCCATCATCCAGGGCATCCTTTTGC |
| | | BIPe | TGATCAGGACGGAGACT |
| | 9 | TSPYN2 | CGCCCTGCAGGTAGAACTG |
| | | F3 | |
| | 10 | TSPYN2 | AGTCCATCATGTAGCCGAGA |
| | | B3 | |
| | 17 | TSPYN2 | CATGAACTGAACGTAAGCCCT |
| | | LF | |
| | 34 | TSPYN2 | GGCCAAAGCTATTATGAGCCA |
| | | LB | |
| TSPY (6) | 15 | bTSPY1- | TGACGCTTCCTCCTCTGATGGTAGCT |
| | | FIB | CTGAGCGTGAGCAA |
| | 16 | bTSPY1- | CGCCATCATCCAGGGCATCCGCTGAT |
| | | BIP | CAGGACGGAGACT |
| | 9 | bTSPY1 | CGCCCTGCAGGTAGAACTG |
| | | F3 | |
| | 10 | bTSPY1 | AGTCCATCATGTAGCCGAGA |
| | | B3 | |
| | 11 | bTSPY1- | TGAACTGAACGTAAGCCCT |
| | | LF | |
| | 12 | bTSPY1- | GGGCCAAAGCTATTATGAGCCA |
| | | LB | |
| 1.715 | 18 | B1.715 | TCCACCCAGTGAGCTCCAAGAGGGAA |
| (set 1) | | FIP | GCAAAGAACCCCGC |
| | 19 | B1.715 | GTCAAGCCTCCTGAGGCGTTAGAGAT |
| | | BIP | GAGGGCCTTCGTC |
| | 20 | B1.715 | CGGGGAGACAGGCATTCA |
| | | F3 | |
| | 21 | B1.715 | GAGAACAACCCCGAGATTGG |
| | | B3 | |
| | 22 | B1.715 | CGTCACAATTCGAGAGCAGA |
| | | LF | |
| | 23 | B1.715 | TGGAGAGAGGTCGCGAGAT |
| | | LB | |
| 1.715e | 24 | B1.715- | TCCACCCAGTGAGCTCCAAGAGTTTT |
| (set 2) | | FIPe | GGAAGCAAAGAACCCCGC |
| | 25 | B1.715 | GTCAAGCCTCCTGAGGCGTTTTTTAG |
| | | BIPe | AGATGAGGGCCTTCGTC |
| | 20 | B1.715 | CGGGGAGACAGGCATTCA |
| | | F3 | |
| | 21 | B1.715 | GAGAACAACCCCGAGATTGG |
| | | B3 | |
| | 22 | B1.715 | CGTCACAATTCGAGAGCAGA |
| | | LF | |
| | 23 | B1.715 | TGGAGAGAGGTCGCGAGAT |
| | | LB | |
| TSPY | 26 | TSPYN3 | TGTTCCTGCTCCTCATCCGCCACCAG |
| (set 7) | | FIP | CTGCTTGCGGAAGA |
| | 27 | TSPYN3 | AGCTAGAGGAGAAGACGGTGGATCCT |
| | | BIP | GGCGCTCACAAGG |
| | 28 | TSPYN3 | GGATCGGGCGGGAGTT |
| | | F3 | |
| | 29 | TSPYN3 | TGCCTGCACGGCATCC |
| | | B3 | |
| | 30 | TSPYN3 | CCTCCACCTCCTCCATGATG |
| | | LF | |
| | 31 | TSPYN3 | AGCAGGGCCAGGAAAGG |
| | | LB | |
| TSPY (8) | 32 | TSPYN3 | TGTTCCTGCTCCTCATCCGCCATTTT |
| | | FIPe | CCAGCTGCTTGCGGAAGA |
| | 33 | TSPYN3 | AGCTAGAGGAGAAGACGGTGGATTTT |
| | | BIPe | CCTGGCGCTCACAAGG |
| | 28 | TSPYN3 | GGATCGGGCGGGAGTT |
| | | F3 | |
| | 29 | TSPYN3 | TGCCTGCACGGCATCC |
| | | B3 | |
| | 30 | TSPYN3 | CCTCCACCTCCTCCATGATG |
| | | LF | |
| | 31 | TSPYN3 | AGCAGGGCCAGGAAAGG |
| | | LB | |
| TSPY (9) | 13 | TSPYN1 | TGACGCTTCCTCCTCTGATGGTGAGC |
| | | FIP | AAAACCGCAGGGCTTA |
| | 14 | TSPYN1 | CGCCATCATCCAGGGCATCCATCAGG |
| | | BIP | ACGGAGACTTGAGG |
| | 5 | TSPYN1 | TCTTGCGCATGAACTGAACG |
| | | LF | |
| | 6 | TSPYN1 | CTGGCTTCTGGGCCAAAG |
| | | LB | |
| TSPY (10) | 15 | TSPYN2 | TGACGCTTCCTCCTCTGATGGTAGCT |
| | | FIP | CTGAGCGTGAGCAA |
| | 16 | TSPYN2 | CGCCATCATCCAGGGCATCCGCTGAT |
| | | BIP | CAGGACGGAGACT |
| | 17 | TSPYN2 | CATGAACTGAACGTAAGCCCT |
| | | LF | |
| | 34 | TSPYN2 | GGCCAAAGCTATTATGAGCCA |
| | | LB | |
| TSPY (11) | 26 | TSPYN3 | TGTTCCTGCTCCTCATCCGCCACCAG |
| | | FIP | CTGCTTGCGGAAGA |
| | 27 | TSPYN3 | AGCTAGAGGAGAAGACGGTGGATCCT |
| | | BIP | GGCGCTCACAAGG |
| | 30 | TSPYN3 | CCTCCACCTCCTCCATGATG |
| | | LF | |
| | 31 | TSPYN3 | AGCAGGGCCAGGAAAGG |
| | | LB | |
| TSPY (12) | 1 | TSPYN1 | TGACGCTTCCTCCTCTGATGGTTTTT |
| | | FIPe | GAGCAAAACCGCAGGGCTTA |
| | 2 | TSPYN1 | CGCCATCATCCAGGGCATCCTTTTAT |
| | | BIPe | CAGGACGGAGACTTGAGG |
| | 5 | TSPYN1 | TCTTGCGCATGAACTGAACG |
| | | LF | |
| | 6 | TSPYN1 | CTGGCTTCTGGGCCAAAG |
| | | LB | |
| TSPY (13) | 7 | TSPYN2 | TGACGCTTCCTCCTCTGATGGTTTTT |
| | | FIPe | AGCTCTGAGCGTGAGCAA |
| | 8 | TSPYN2 | CGCCATCATCCAGGGCATCCTTTTGC |
| | | BIPe | TGATCAGGACGGAGACT |
| | 17 | TSPYN2 | CATGAACTGAACGTAAGCCCT |
| | | LF | |
| | 34 | TSPYN2 | GGCCAAAGCTATTATGAGCCA |
| | | LB | |
| TSPY (14) | 32 | TSPYN3 | TGTTCCTGCTCCTCATCCGCCATTTT |
| | | FIPe | CCAGCTGCTTGCGGAAGA |
| | 33 | TSPYN3 | AGCTAGAGGAGAAGACGGTGGATTTT |
| | | BIPe | CCTGGCGCTCACAAGG |
| | 30 | TSPYN3 | CCTCCACCTCCTCCATGATG |
| | | LF | |
| | 31 | TSPYN3 | AGCAGGGCCAGGAAAGG |
| | | LB | |
| HSFY | 44 | HSFY_F | TGTCTCAAATATCTTCATTAGTGC |
| | | 3 | |
| | 45 | HSFY_B | GGTGGTTAGGTAAGTCACAT |
| | | 3 | |
| | 46 | HSFY_F | GCGCTCTCTCTACTAATTTCTGGAAA |
| | | IP | GATAACAAGAAGCATGTTAAAGC |
| | 47 | HSFY_B | TTCAGCCTCCACAAGTTTACCTGCAT |
| | | IP | TTGTATTAGTGACAATCTGG |
| | 48 | HSFY_L | CAGAGTTACGATCATCTTCGTTGA |
| | | F | |
| | 49 | HSFY_L | GTGCCTTTCATACAAAAGCCT |
| | | B | |

Additional desired traits may include a health trait, a reproductive trait, a disease resistance trait, an anatomical trait, the presence of a gene edit, a value from a genotype-based value model such as an overall Estimated Breeding Value (EBV) or other genomic index, or the sex of the embryo. The target locus can encode a protein involved in the health, welfare, growth, reproduction, or well-being of the animal. Alternatively, or in addition, the target locus can encode a protein involved in production of milk or meat (via expansion of the animal's muscle mass). Alternatively, or in addition, the target locus can be on mitochondrial DNA (mtDNA).

Exemplary bovine traits include polled (lack of horns), sterility or fertility, milk production, growth (which increases meat production), fat production, conception rates, stillborn rates, calving ease, or content of produced milk such as the amount of protein or the amount of fat. Further bovine traits can include backfat thickness, intramuscular fat, ultrasound loin muscle area, loin muscle area and intramuscular fat content, chest circumference, withers height, body length, hip height, rump length, and heart girth. Further traits include high altitude adaptation and response to hypoxia (DCAF8, PPP1R12A, SLC16A3, UCP2, UCP3, TIGAR), cold acclimation (AQP3, AQP7, HSPB8), body size and stature (PLAG1, KCNA6, NDUFA9, AKAP3, C5H12orf4, RAD51AP1, FGF6, TIGAR, CCND2, CSMD3), resistance to disease and bacterial infection (CHI3L2, GBP6, PPFIBP1, REP15, CYP4F2, TIGD2, PYURF, SLC10A2, FCHSD2, ARHGEF17, RELT, PRDM2, KDM5B), reproduction (PPP1R12A, ZFP36L2, CSPP1), milk yield and components (NPC1L1, NUDCD3, ACSS1, FCHSD2, Kappa Casein and Beta Casein, CSN2, CSN3), growth and feed efficiency (TMEM68, TGS1, LYN, XKR4, FOXA2, GBP2, GBP5, FGD6), and polled phenotype (URB1, EVA1C, haplotype TP). Exemplary target genes can include PRLR, NANOS2, Deadend (Dnd), APAF1, SMC2, GART, TFB1M, SIRT1, SIRT2, LPL, CRTC2, SIX4, UCP2, UCP3, URB1, EVA1C, TMEM68, TGS1, LYN, XKR4, FOXA2, GBP2, GBP5, FGD6, NPC1L1, NUDCD3, ACSS1, FCHSD2, PPP1R12A, ZFP36L2, CSPP1, CHI3L2, GBP6, PPFIBP1, REP15, CYP4F2, TIGD2, PYURF, SLC10A2, FCHSD2, ARHGEF17, RELT, PRDM2, KDM5B, PLAG1, KCNA6, NDUFA9, AKAP3, C5H12orf4, RAD51AP1, FGF6, TIGAR, CCND2, CSMD3, AQP3, AQP7, HSPB8, DCAF8, PPP1R12A, SLC16A3, AMPK, PKM2, PDH, LDHA, LDHC, and ZBTB.

Exemplary porcine traits include meat production traits such as growth rate, backfat depth, muscle pH, purge loss, muscle color, firmness, marbling scores, intramuscular fat percentage, tenderness, average daily gain, average daily feed intake, feed efficiency, back fat thickness, loin muscle area, and lean percentage. Exemplary health traits include the absence of undesirable physical abnormalities or defects (like scrotal ruptures), improvement of feet and leg soundness, resistance to specific diseases or disease organisms, or general resistance to pathogens. Further health traits can include melanotic skin tumors, dermatosis vegetans, abnormal mamae, shortened vertebral column, kinky tail, rudimentary tail, hairlessness, woolly hair, hydrocephalus, tassels, legless, three-legged, syndactyly, polydactyly, pulawska factor, heterochromia iridis, congenital tremor a iii, congenital tremor a iv, congenital ataxia, hind leg paralysis, bentleg, thickleg, malignant hyperthermia, hemophilia (von Willebrand's disease), leukemia, hemolytic disease, edema, acute respiratory distress (“barker”), rickets, renal hypoplasia, renal cysts, uterus aplasia, porcine stress syndrome (pss), halothane (hal), dipped shoulder (humpy back, kinky back, kyphosis), hyperostosis, mammary hypoplasia, undeveloped udder, and epitheliogenesis imperfecta. Exemplary target sequences include ANP32, ANPEP, TMPRSS1, TMPRSS2, NANOS2, CD163, Melanocortin-4 receptor (MC4R), HMGA, IGF2, E. coli F4ab/ac, HAL, RN, Mxl, BAT2, EHMT2, AMPK, PKM2, PDH, LDHA, LDHC, and ESR. The present method is especially useful for determining the sex of the embryo by detecting loci on the Y chromosome.

In certain embodiments, LAMP amplification is used to ascertain the sex of livestock embryos. For example, but without limitation, a test can discriminate between the sexes by selecting a locus that enables detection of the presence or absence of the Y chromosome. Several Y loci are possible, including SRY, TSPY, S4 (a tandem repeat sequence published in Kageyama, S. et al., J. Vet. Med. Sci., 2004, 66, 509-514), DDX3Y, EIFIAY, HSFY, USP9Y, ZFY, ZnFY, and ZRSR2Y. The present inventors chose to examine loci in the bovine TSPY gene because of its high copy number (>250 copies) and lack of cross-reaction with female DNA.

TABLE 2
Exemplary nucleic acid sequences
associated with the Y chromosome.

| Gene | GenBank Acc. No. |
|---|---|
| TSPY | NM_001244608.1 (SEQ ID NO: 50), |
| | AF042179.1 (SEQ ID NO: 51) |
| SRY | MN727885.1 (SEQ ID NO: 52), |
| | MH322033.1 (SEQ ID NO: 53) |
| DDX3Y | GQ259591.1 (SEQ ID NO: 54), |
| | KU705630.1 (SEQ ID NO: 55) |
| EIF1AY | NM_001145757.1 (SEQ ID NO: 56), |
| | NM_001244349.1 (SEQ ID NO: 57) |
| HSFY | JF281100 (SEQ ID NO: 58), KT371431 |
| | (SEQ ID NO: 59) |
| USP9Y | MF541997.1 (SEQ ID NO: 60), FJ627275.1 |
| | (SEQ ID NO: 61) |
| ZFY | AF032867.1 (SEQ ID NO: 62), |
| | AH011262.2 (SEQ ID NO: 63) |
| ZRSR2Y | JX899373.1 (SEQ ID NO: 64) |
| ZNF280AY | HQ014564 (SEQ ID NO: 65) |
| ZNF280BY | NM_001078120 (SEQ ID NO: 66) |

Exemplary nucleic acid sequences associated with traits such as pathogen resistance, fertility, lactation, or native traits that support more rapid growth or feed efficiency in a non-human animal subject (e.g., cattle, pigs, or fish) include, but are not limited to:

TABLE 3
Exemplary Somatic
Nucleic Acid Sequences

| Gene | GenBank Acc. No. |
|---|---|
| DCAF8 | NM_001206419.2 |
| SLC16A3 | NM_001109980.3 |
| TIGAR | NM_001076370.1 |
| Deadend (Dnd) | JN712911.1 |
| CD163 | NM_213976.1 |
| | XM_021091121.1 |
| | GQ184570.1 |
| ANP32 | XM_003121759 |
| (ANP32A and ANP32B) | XM_021066477.1 |
| | NM_001195019.1 |
| | NM_001035074.1 |
| ANPEP | XM_005653524.3 |
| | XM_021098304.1 |
| | NM_001075144.1 |
| TMPRSS2 | NM_001386131.1 |
| | NM_001081585.1 |
| NANOS | XM_001928298 |
| | XM_003127232.1 |
| | XM_005661246.1 |
| | XM_005225796 |
| Melanocortin-4 receptor | NM_214173.1 |
| (MC4R) | NM_174110.1 |
| PRLR | NM_001039726.2 |
| | NM_174155.3 |
| | NM_001001868.1 |
| HMGA | XM_005663957.3 |
| | XM_021091958.1 |
| | XM_002687648.5 |
| IGF2 | NM_213883.2 |
| | NM_001367627.1 |
| MUC13 | NM_001105293.1 |
| HAL | XM_001925061.4 |
| Mx1 | NM_214061.2 |
| BAT2 | NM_001114675.1 |
| EHMT2 | NM_001101823.1 |
| | NM_001206263.2 |
| PRDM1 | NM_001192936.1 |
| | XM_005659341.3 |
| PRDM14 | NM_001191264.1 |
| | XM_021090784.1 |
| ESR | NM_214220.1 |
| APAF1 | NM_001191507.1 |
| | XM_021093024.1 |
| SMC2 | XM_021066949.1 |
| | XM_024996295.1 |
| GART | NM_001040473.2 |
| | XM_021070945.1 |
| TFB1M | NM_001076896.2 |
| | NM_001128475.1 |
| SIRT1 | NM_001145750.2 |
| | NM_001192980.3 |
| SIRT2 | XM_021093185.1 |
| | NM_001113531.1 |
| LPL | NM_001075120.1 |
| | NM_214286.1 |
| CRTC2 | XM_005663405.3 |
| | NM_001076250.1 |
| SIX4 | XM_024998078.1 |
| | NM_001244614.1 |
| UCP2 | NM_001033611.2 |
| | NM_214289.1 |
| UCP3 | NM_174210.1 |
| | NM_214049.1 |
| URB1 | NM_001205980.1 |
| EVA1C | XM_024988259.1 |
| TMEM68 | NM_001076009.1 |
| TGS1 | XM_015474433.2 |
| LYN | XM_025001638.1 |
| XKR4 | XM_002692650.4 |
| FOXA2 | XM_025001047.1 |
| FGD6 | XM_005206128.4 |
| NPC1L1 | XM_002686890.5 |
| NUDCD3 | XM_005205595.3 |
| ACSS1 | XM_025000325.1 |
| FCHSD2 | XM_024975871.1 |
| PPP1R12A | XM_024991981.1 |
| ZFP36L2 | NM_001191191.1 |
| CSPP1 | XM_025001590.1 |
| CHI3L2 | XM_024990025.1 |
| GBP6 | NM_001075995.1 |
| GBP5 | HF564747.1 |
| PPFIBP1 | XM_010805392.3 |
| REP15 | XM_005206922.4 |
| CYP4F2 | XM_010806565.3 |
| TIGD2 | XM_005207816.4 |
| PYURF | NM_001038102.2 |
| SLC10A2 | XM_025000165.1 |
| ARHGEF17 | NM_001282586.1 |
| RELT | XM_002693451.5 |
| PRDM2 | XM_015475202.2 |
| KDM5B | XM_024976577.1 |
| PLAG1 | XM_005215434.3 |
| KCNA6 | NM_001206413.3 |
| NDUFA9 | NM_205817.1 |
| AKAP3 | XM_015471316.2 |
| C5H12orf4 | XM_005207206.4 |
| RAD51AP1 | NM_001076403.1 |
| FGF6 | NM_001192400.1 |
| CCND2 | XM_024992177.1 |
| CSMD3 | NM_001105499.2 |
| AQP3 | NM_001079794.1 |
| AQP7 | XM_024995941.1 |
| HSPB8 | NM_001014955.1 |

In various embodiments, the trait can be a change in a gene that confers resistance to a pathogen or disease. In some embodiments, the pathogen is a virus such as PRRS virus, African Swine Fever virus, HiNi virus, a coronavirus, Salmon alphavirus, infectious pancreatic necrosis virus (IPNV), infectious salmon anaemia virus, piscine myocarditis virus (PMCV), aquareovirus, infectious hematopoietic necrosis virus, viral hemorrhagic septicemia virus, Bovine viral diarrhea virus, Bovine leukemia virus, Bovine herpesvirus, Lumpky skin disease virus, Classical swine fever virus, Nipah virus, Swine vesicular disease virus, Transmissible gastroenteritis virus of swine, West Nile fever virus, Vesicular stomatitis virus, Japanese encephalitis virus, Rinderpest virus, Rift Valley fever virus, Rabies virus, Foot-and-mouth disease virus, aquabirnaviruses, betanodaviruses, nervous necrosis virus, epizootic hematopoietic necrosis virus, European catfish virus, and Pseudorabies virus.

In certain embodiments, the pathogen is a bacterium, such as Mannheimia haemolytica, Escherichia coli, Salmonella spp., Listeria monocytogenes, Clostridium spp., Campylobacter, Yersinia enterocolitica, Mycobacterium avium, Moraxella bovis, Brucella abortus, Piscirickettsia salmonis, Streptococcus agalactiae, Aeromonas salmonicida, Leptospira spp., Pasteurella multocida, Mycoplasma mycoides, Trueperella pyogenes, Mycoplasma bovis, Mycobacterium bovis, Chlamydophila abortus, Coxiella burnetii, and Francisella tularensis. In other embodiments, the pathogen is a protozoa, such as Neospora caninum, Sarcocystis spp., Tritrichomonas foetus, Neoparamoeba perurans, Cryptosporidium parvum, and Giardia lamblia.

In various configurations, the pathogen can be Lepeophtheirus salmonis, Caligus clemensi, Caligus rogercresseyi. Yersinia ruckeri, Edwardsiella ictalurid, Flavobacterium psychrophilum, Flavobacterium branchiophilum, Piscirickettsia salmonis, or Francisella noatunensis.

“Genotype-based value models” include any estimate of an animal's value based on a model that uses genotype data as an input. A very simple example would be a presence/absence genetic marker for a trait of interest-presence indicates one value while absence indicates a different value. Several other genotype-based value models follow. An animal's breeding value can be defined as its genetic merit for each trait. While it is not possible to determine an animal's true breeding value, it is possible to estimate it. These estimates of an animal's true breeding value are called estimated breeding values (EBVs). EBVs are expressed as the difference between an individual animal's genetics and the genetic base to which the animal is compared. EBVs are reported in the units in which the measurements are taken (e.g., kilograms for the weight EBVs). Thus, a value of +12 kg for 400-day weight means the animal is genetically superior by 12 kg at 400 days compared with the genetic base of the relevant cattle population. On average, half of this difference will be passed on to the animal's progeny. Another method of estimating breeding value is using an animal's genomic makeup. Genomic selection refers to selection decisions based on genomic estimated breeding values (GEBV). To calculate GEBV, first a prediction equation based on a large number of DNA markers, such as SNP (Single Nucleotide Polymorphisms) markers, is derived. The effects of these markers are estimated in a reference population in which animals are both phenotyped and genotyped. In subsequent generations, animals can be genotyped for the markers and the effects of the genotypes summed across the whole genome to predict the GEBV. Cattle breeding associations such as the Council on Dairy Cattle Breeding (CDCB) have established reference populations that are used to calculate genomic estimated breeding values. CDCB further creates selection indices based on genomic predictions for various traits, including Net Merit (NM$) and Cheese Merit (CM$), which estimate the additional revenue a cow will generate over her lifetime relative to a theoretical base animal. The CDCB adjusts these calculations from time to time in conjunction with the USDA. Current calculations are available on the group's website at uscdcb.com. The SNP markers used are known in the art and can be leveraged to calculate genomic estimated breeding values for a particular trait, and then selection index values based on those traits.

Calculation of EBVs for beef cattle is less centralized, as different breed associations and genetics companies each have their own indices. For example, the American Angus Association provides genome enhanced progeny differences-which take into account data on a particular animal, its relatives, and genomic data on various traits to predict the difference between the animal and its parents. Unlike in the dairy industry, the SNPs driving a particular trait are proprietary; however, the present teachings can be used with any given genomic marker provided that the effect is known.

In contrast with bovine breeding, porcine selection is even less centralized. Each breeder uses their own custom index depending on their breeding goals. Many rely on the “best linear unbiased prediction” or BLUP statistical model. Most software for calculating the trait index is customized for a specific breeder, but publicly available programs are known in the art. There are several art recognized statistical models, which also include GBLUP, which brings genomic markers into the model (Stock, J., Front. Genet. 2020; 11: 568). These models are used to predict Estimated Breeding Values (EBVs; Published US application 2005/0221322 by Fox et al.). It is within the knowledge of the skilled artisan to choose the appropriate gene panel for such a model.

The methods of the present teachings can be performed using a kit comprising the needed reagents. The kit can comprise a set of primers directed to a desired locus, an indicator molecule, and amplification reagents needed for amplification. In general, these amplification reagents can comprise dNTPs, a DNA polymerase, buffer, or a combination thereof. For PCR amplification, the DNA polymerase can be TAQ or any other thermostable DNA polymerase. Magnesium ion sources such as MgCl₂ can be provided as cofactors for a PCR reaction. For LAMP amplification, the DNA polymerase can be a strand displacement DNA polymerase such as Bst DNA polymerase or Bsu DNA polymerase. A number of variants of these polymerases are commercially available, such as from New England Biolabs or Varigen.

The components of the kit can be provided as individual components, or as a premade master mix. The premade master mix can contain all of the components, or some components can be provided separately. For example, some primers will form primer dimers when allowed to interact with polymerase in the absence of template. Therefore, the primers may be provided in a separate tube to be added with the template. As discussed supra, the master mix may be provided in a liquid form or a lyophilized form. The premade master mix can be in a single tube, or can be in individual reaction tubes ready for addition of cell-free template DNA.

In various embodiments, a kit for use in the present teachings can comprise reagents for whole genome amplification and reagents for sequencing or genotyping the amplified DNA. The reagents for whole genome amplification may include random oligo primers of various lengths for primer extension amplification or partially degenerate primers for degenerate oligonucleotide primed PCR. In some configurations, the primers can be 15-20 base pairs in length. For either of these primers, the kit would also include a thermostable DNA polymerase, dNTPs, and PCR buffer, either separately or in a master mix. Alternatively, the kit may contain random hexamers and a displacement DNA polymerase such as φ29 DNA polymerase or Bst polymerase for multiple displacement amplification. Alternatively, the kit may contain bar coded primers designed for amplifying loci of particular interest—thus amplifying the needed regions rather than the whole genome. These regions can then be subjected to standard next generation sequencing reactions.

The kit can further comprise primer sets for a panel of markers. The primers may be provided in separate tubes, or each set of primers may be pre-arrayed in a multi-well vessel, such as a 24 or 96 well block or plate. The vessel can be configured to be used on a pipetting robot such as, but without limitation, a Biomek robot from Beckman-Coulter. Conditioned media comprising cell-free DNA can then be added to each well either via hand pipetting (using a single or multi-channel pipette, although the latter would be preferred for ergonomic reasons) or via automation. The results can be read visually or via an automatic plate reader. The plate reader can then feed data into a computer, which can use the results to calculate a genotype-based value model

In some configurations, the panel of markers can comprise single nucleotide polymorphisms (SNPs) selected from the industry standard panel of SNPs used for genotype testing (see Bovine HapMap Consortium, Science, 2009, 324, 528-532). In various configurations, a primer can include a DNA bar code for multiplexing reactions. Products from these multiplexed reactions are then sequenced, and the exact product can be identified by the DNA barcode sequence. In this manner, multiple LAMP reactions may be performed in the same vessel, allowing for LAMP based SNP genotyping.

The reagents for sequencing the DNA are known in the art, and are dependent on the application. Appropriate SNP arrays for genotyping are also known in the art and listed supra.

In various configurations, the amplification reactions can include set of probes comprising a “sink” primer and a labeled primer with high affinity for a desirable SNP. A kit comprising primers for a panel of markers can include a plurality of labelled high affinity probes with sink probes that can optionally be differentially labeled. In various configurations, the “loop” primers can be short primers (around 11 bp) that bind specifically to the desired sequence in the trait, which enhances the speed at which the desired allele is amplified. This amplification can be monitored by a fluorescent label-quencher complex on the primer, wherein the quencher is cleaved when the primer is incorporated into an amplicon.

In various configurations, the amplification reaction agents can include a probe comprising a fluorescent or colorimetric moiety and a quencher. When the probe is incorporated into the LAMP product, the quencher is cleaved, producing a change in fluorescence. Probes with different colored labels (e.g., TEX, Cy5, and HEX) can be placed in the same vessel to allow for multiple LAMP reactions to occur in the same well. The results can then be read in a plate reader or real time PCR machine.

In various embodiments, the present teachings include the following non-limiting aspects:

In various embodiments, the present teachings provide a method of making an animal or a cell line having a desirable trait from an in vitro produced embryo comprising: combining male and female gametes in vitro to form an embryo or producing a parthenogenetic embryo; culturing the embryo for a period of time sufficient for cell-free DNA to accumulate in the medium; adding medium from the embryo culture to an amplification reaction mix comprising primers targeted to a trait-related locus and at least one turbidimetric, colorimetric, or fluorescent indicator; amplifying the cell-free DNA; determining that the primers amplify a locus for the desirable trait or determining that the primers do not amplify a locus for an undesirable trait based on a signal from the at least one turbidimetric, colorimetric, or fluorescent indicator; selecting the embryo; and implanting the selected embryo into a surrogate mother or using cells from the embryo to create a cell line. In various configurations, the amplifying the DNA can be by loop-mediated isothermal amplification (LAMP). In various configurations, the period of time can be 3-9 days. In various configurations, the period of time can be 7 days. In various configurations, the trait can be a health trait, a reproductive trait, a disease resistance trait, an anatomical trait, a desired gene edit, or sex of the embryo. In various configurations, the trait can be sex of the embryo and the locus is on the Y chromosome. In various configurations, the locus can be TSPY. In various configurations, the primers can comprise a set of primers consisting of: SEQ ID NO: 1-6, SEQ ID NO: 7-12, SEQ ID NO: 3-6, and 13-14, SEQ ID NO: 9-10, 15-17, and 34, SEQ ID NO: 7-10, 17, and 34, SEQ ID NO: 9-12 and 15-16, SEQ ID NO: 26-31, SEQ ID NO: 28-33, SEQ ID NO: 5-6 and 13-14, SEQ ID NO: 15-17 and 34, SEQ ID NO: 26-27 and 30-31, SEQ ID NO: 1-2 and 5-6, SEQ ID NO: 7-8, 17, and 34, or SEQ ID NO: 30-33. In various configurations, the locus can be HSFY or TSPY. In various configurations, the primers can comprise a set of primers consisting of SEQ ID NO: 44-49. In various configurations, the embryo desired can be female and the embryo can be selected if the at least one turbidimetric, colorimetric, or fluorescent indicator indicates that the primers do not amplify TSPY or HSFY. In various configurations, the embryo desired can be male and the embryo can be selected if the at least one turbidimetric, colorimetric or fluorescent indicator indicates that the primers do amplify TSPY or HSFY. In various configurations, the at least one turbidimetric, colorimetric, or fluorescent indicator can be a pH indicator or a fluorescent indicator. In various configurations, the at least one turbidimetric, colorimetric, or fluorescent indicator can be selected from the group consisting of phenol red, bromothymol blue, thymol blue, bromocresol purple, thymolphthalein, phenolphthalein, neutral red, brilliant yellow, cresol red, 4-(2-pyridylazo) resorcinol (PAR), and hydroxynaphthol blue. In various configurations, the at least one turbidimetric, colorimetric, or fluorescent indicator can comprise phenol red. In various configurations, the at least one turbidimetric, colorimetric, or fluorescent indicator can be an intercalating dye. In various configurations, the at least one turbidimetric, colorimetric, or fluorescent indicator is selected from the group consisting of SYTO 9, SYTO 13, SYTO 16, SYTO 64, SYTO 82, Boxto, Miami Green, Miami Yellow, Miami Orange, YOPRO 1, SYTO 62, TOPRO 3, SYTO 60, EvaGreen, POPO 3, DCS1, SYBR Green I, BOBO 3, Pico 488, VIC, and TOTO 3. In various configurations, the at least one turbidimetric, colorimetric, or fluorescent indicator can be SYTO 82. In various configurations, the method can further comprise determining a value based on a genotype-based value model. In various configurations, the locus can be a target of gene editing for a desired edit, and the primers are directed to the desired edit.

In various embodiments, the present teachings provide for a kit comprising: amplification reagents, a set of primers directed to a particular locus, and at least one turbidimetric, colorimetric, or fluorescent indicator molecule. In various configurations, the amplification reagents can comprise a DNA polymerase. In various configurations, the amplification reagents can comprise a nucleic acid polymerase selected from the group consisting of a TAQ polymerase, Bsu DNA polymerase, and Bst DNA Polymerase. In various configurations, the amplification reagents can comprise Bst Polymerase and the set of primers can comprise four primers directed to the particular locus. In various configurations, the amplification reagents can comprise Bst Polymerase and the set of primers can comprise six primers directed to the locus. In various configurations, the at least one turbidimetric, colorimetric or fluorescent indicator can be selected from the group consisting of phenol red, bromothymol blue, thymol blue, bromocresol purple, thymolphthalein, phenolphthalein, neutral red, brilliant yellow, cresol red, 4-(2-pyridylazo) resorcinol (PAR), and hydroxynaphthol blue. In various configurations, the at least one turbidimetric, colorimetric, or fluorescent indicator can comprise phenol red. In various configurations, the at least one turbidimetric, colorimetric or fluorescent indicator can be selected from the group consisting of SYTO 9, SYTO 13, SYTO 16, SYTO 64, SYTO 82, Boxto, Miami Green, Miami Yellow, Miami Orange, YOPRO 1, SYTO 62, TOPRO 3, SYTO 60, EvaGreen, POPO 3, DCS1, SYBR Green I, BOBO 3, Pico 488, VIC, and TOTO 3. In various configurations, the at least one turbidimetric, colorimetric or fluorescent indicator can be SYTO 82. In various configurations, the kit can be for sex determination of an embryo, and the particular locus can be TSPY or HSFY. In various configurations, the particular locus can be TSPY, the amplification reagents can comprise Bst Polymerase, the set of primers can have sequences SEQ ID NO: 1-6, and at least one turbidimetric, colorimetric, or fluorescent indicator can comprise phenol red. In various configurations, the particular locus can be HSFY, the amplification reagents can comprise Bst Polymerase, the set of primers can have sequences SEQ ID NO: 44-49, and the at least one turbidimetric, colorimetric, or fluorescent indicator can comprise SYTO 82.

In various embodiments, a method of selecting an in vitro produced embryo for implantation can comprising: combining male and female gametes in vitro to form an embryo; culturing the embryo for a period of time sufficient for cell-free DNA to accumulate in medium; adding the medium from the embryo culture to a whole genome amplification reaction mix; amplifying the cell-free DNA to obtain an amplified genome; determining the genotype of the embryo; and implanting the embryo. In various configurations, the method can further comprise calculating a value from a genotype-based value model and selecting the embryo based on the value from the genotype-based value model. In various configurations, the whole genome amplification mix can be selected from the group consisting of a primer extension preamplification mix, a degenerate oligonucleotide primed-polymerase chain reaction mix, and a multiple displacement amplification mix. In various configurations, the embryo can be a non-human mammalian embryo. In various configurations, the embryo can be a bovine embryo or a porcine embryo. In various configurations, the embryo can be a Bos taurus, Bos indicus, or a Sus scrofa embryo. In various configurations, the determining the genotype of the embryo can comprise sequencing the amplified genome using a SNP array.

In various embodiments, a kit of the present teachings can comprise: a whole genome amplification reaction mix; and a SNP array. In various configurations, the whole genome amplification mix can be selected from the group consisting of a primer extension preamplification mix, a degenerate oligonucleotide primed-polymerase chain reaction mix, and a multiple displacement amplification mix.

In various embodiments, the present teachings provide for method of making a female animal or a female cell line from an in vitro produced embryo comprising: combining male and female gametes in vitro to form an embryo or producing a parthenogenetic embryo; culturing the embryo or parthenogenetic embryo for a period of time sufficient for cell-free DNA to accumulate in the medium; adding medium from the embryo culture or parthenogenetic embryo culture to a LAMP amplification reaction mix comprising a set of primers targeted to a TSPY locus comprising the nucleic acid sequences consisting of SEQ ID NO: 1-6, SEQ ID NO: 7-12, SEQ ID NO: 3-6, and 13-14 SEQ ID NO: 9-10, 15-17, and 34, SEQ ID NO: 7-10, 17, and 34, SEQ ID NO: 9-12 and 15-16, SEQ ID NO: 26-31, SEQ ID NO: 28-33, SEQ ID NO: 5-6 and 13-14, SEQ ID NO: 15-17 and 34, SEQ ID NO: 26-27 and 30-31, SEQ ID NO: 1-2 and 5-6, SEQ ID NO: 7-8, 17, and 34, or SEQ ID NO: 30-33 and a colorimetric indicator; amplifying the cell-free DNA; determining that the primers do not amplify the TSPY locus based on a signal from the colorimetric indicator; selecting the embryo or parthenogenetic embryo; and implanting the selected embryo into a surrogate mother or using cells from the embryo or parthenogenetic to create a cell line. In various configurations, the period of time is 7 days.

In various embodiments, the present teachings can provide for a method of making a female animal or a female cell line from an in vitro produced embryo comprising: combining male and female gametes in vitro to form an embryo or producing a parthenogenetic embryo; culturing the embryo or parthenogenetic embryo for a period of time sufficient for cell-free DNA to accumulate in the medium; adding medium from the embryo culture to a LAMP amplification reaction mix comprising a set of primers targeted to an HSFY locus comprising the nucleic acid sequences consisting of SEQ ID NO: 44-49 and a fluorescent indicator; amplifying the cell-free DNA; determining that the primers do not amplify the HSFY locus based on a signal from the fluorescent indicator; selecting the embryo or parthenogenetic embryo; and implanting the selected embryo into a surrogate mother or using cells from the embryo or parthenogenetic embryo to create a cell line. In various configurations, the period of time can be 7 days.

In various embodiments, the present teachings can provide for, method of making a male animal or a male cell line from an in vitro produced embryo comprising: combining male and female gametes in vitro to form an embryo or producing a parthenogenetic embryo; culturing the embryo or parthenogenetic embryo for a period of time sufficient for cell-free DNA to accumulate in the medium; adding medium from the embryo culture or parthenogenetic embryo culture to a LAMP amplification reaction mix comprising a set of primers targeted to a TSPY locus comprising the nucleic acid sequences consisting of SEQ ID NO: 1-6, SEQ ID NO: 7-12, SEQ ID NO: 3-6, and 13-14, SEQ ID NO: 9-10, 15-17, and 34, SEQ ID NO: 7-10, 17, and 34, SEQ ID NO: 9-12 and 15-16, SEQ ID NO: 26-31, SEQ ID NO: 28-33, SEQ ID NO: 5-6 and 13-14, SEQ ID NO: 15-17 and 34, SEQ ID NO: 26-27 and 30-31, SEQ ID NO: 1-2 and 5-6, SEQ ID NO: 7-8, 17, and 34, and SEQ ID NO: 30-33 and a colorimetric indicator; amplifying the cell-free DNA; determining that the primers amplify the TSPY locus based on a signal from the colorimetric indicator; selecting the embryo or parthenogenetic embryo; and implanting the selected embryo into a surrogate mother or using cells from the embryo or parthenogenetic embryo to create a cell line. In various configurations, the period of time can be 7 days.

In various embodiments, a method of making a male animal or a male cell line from an in vitro produced embryo can comprise: combining male and female gametes in vitro to form an embryo or producing a parthenogenetic embryo; culturing the embryo or parthenogenetic embryo for a period of time sufficient for cell-free DNA to accumulate in the medium; adding medium from the embryo culture or parthenogenetic embryo culture to a LAMP amplification reaction mix comprising a set of primers targeted to a HSFY locus comprising the nucleic acid sequences consisting of SEQ ID NO: 44-49 and a fluorescent indicator; amplifying the cell-free DNA; determining that the primers amplify the HSFY locus based on a signal from the fluorescent indicator; selecting the embryo; and implanting the selected embryo into a surrogate mother or using cells from the embryo or parthenogenetic embryo to create a cell line. In various configurations, the period of time can be 7 days.

In various embodiments, a method of making an animal or a cell line having a desirable trait from an in vitro produced embryo can comprise: combining male and female gametes in vitro to form an embryo or producing a parthenogenetic embryo; culturing the embryo or parthenogenetic embryo for a period of time sufficient for cell-free DNA to accumulate in the medium; adding medium from the embryo culture to an amplification reaction mix comprising primers targeted to a trait-related locus and at least one turbidimetric, colorimetric, or fluorescent indicator; amplifying the cell-free DNA; determining that the primers do not amplify a locus for an undesirable trait based on a signal from the at least one turbidimetric, colorimetric, or fluorescent indicator; selecting the embryo or parthenogenetic embryo; and implanting the selected embryo into a surrogate mother and gestating until birth or using cells from the embryo to create a cell line.

In various embodiments, a method of making an animal or a cell line having a desirable trait from an in vitro produced embryo can comprise: combining male and female gametes in vitro to form an embryo or producing a parthenogenetic embryo; culturing the embryo or parthenogenetic embryo for a period of time sufficient for cell-free DNA to accumulate in the medium; adding medium from the embryo culture or parthenogenetic embryo culture to an amplification reaction mix comprising primers targeted to a trait-related locus and at least one turbidimetric, colorimetric, or fluorescent indicator; amplifying the cell-free DNA; determining that the primers amplify a locus for the desirable trait; selecting the embryo or parthenogenetic embryo; and implanting the selected embryo into a surrogate mother or using cells from the embryo or parthenogenetic embryo to create a cell line.

In various embodiments, a method of selecting an in vitro produced embryo for creating a cell line can comprise: combining male and female gametes in vitro to form an embryo or producing a parthenogenetic embryo; culturing the embryo or parthenogenetic embryo for a period of time sufficient for cell-free DNA to accumulate in the medium; adding medium from the embryo culture or parthenogenetic embryo culture to a whole genome amplification reaction mix; amplifying the cell-free DNA to obtain an amplified genome; determining the genotype of the embryo or parthenogenetic embryo; and using cells from the embryo or parthenogenetic embryo to create a cell line. In various configurations, the determining the genotype of the embryo or parthenogenetic embryo can comprises sequencing the amplified genome using a SNP array.

In various configurations, the present teachings include an embryo produced according to the methods of the present teachings.

In various configurations, the embryo can be a non-human embryo.

### EXAMPLES

The present teachings include descriptions provided in the Examples that are not intended to limit the scope of any claim or embodiment. The following non-limiting examples are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

### Example 1

This example illustrates validation of differential amplification between male and female DNA using colorimetric assay.

A mixture of six primers designed to amplify the TSPY locus was made as follows:

TABLE 4

| | SEQ | 10× | 1× |
|---|---|---|---|
| | ID | Concentration | Concentration |
| Primer | NO: | (Stock) | (Final) |
| TSPYN1_FIPe | 1 | 16 μM | 1.6 μM |
| TSPYN1_BIPe | 2 | 16 μM | 1.6 μM |
| TSPYN1_F3 | 3 | 2 μM | 0.2 μM |
| TSPYN1_B3 | 4 | 2 μM | 0.2 μM |
| TSPYNI_LF | 5 | 4 μM | 0.4 μM |
| TSPYN1_LB | 6 | 4 μM | 0.4 μM |

The following were then mixed together: 12.5 μl of WARMSTART® Colorimetric LAMP 2× Master Mix (New England Biolabs, Ipswich, MA), 2.5 μl of the LAMP primer mix (10× stock), and 10 ng/μl of known male DNA, female DNA, or water in 2.5 μl of in vitro culture medium 2 (IVC2). The mix was then incubated at 67.6° C. for 30 minutes. The tube with male DNA turned yellow, the tubes with female DNA and with water remained pink. These results indicate that TSPY is amplified only from male DNA.

### Example 2

This example illustrates the amplification of cell-free DNA in culture using colorimetric assay.

Bovine embryos were cultured in embryo culture medium.

18 blastocysts were selected. An amplification mix was created by adding 12.5 μl of WARMSTART® Colorimetric LAMP 2× Master Mix (New England Biolabs, Ipswich, MA), 2.5 μl of a LAMP primer mix was created using the ratios in Example 1, but with primers targeting the B1.715 bovine autosomal genome locus (SEQ ID NOs: 20-25), and 2.5 μl of media from the cultures at day 7. Controls with water and bacterial plasmid DNA instead of culture media were also prepared. The mix was then incubated at 61° C. for 30 minutes. All of the tubes with blastocyst culture media turned yellow, indicating the presence and amplification of cell-free DNA.

### Example 3

This example illustrates use of LAMP to determine the sex of bovine blastocysts.

1000 bovine zygotes will be cultured in embryo culture medium.

300-400 blastocysts will be selected. The following will be mixed together: 12.5 μl of WARMSTART® Fluorescent LAMP 2× Master Mix (New England Biolabs, Ipswich, MA), 2.5 μl of the LAMP primer mix as described in Example 1, and 2.5 μl media removed from the blastocyst cultures at day 7. The mix will be incubated at 61° C. for 90 minutes. The tubes with culture from putative male blastocysts will turn yellow, the tubes from cultures with putative female blastocysts will remain pink. For validation purposes, sex determination will be confirmed by real time PCR analysis.

### Example 4

This example illustrates selection of embryos for female replacement heifers.

A dairy farmer wants to make elite replacement heifers for their herd. They aspirate oocytes from three of their heifers and combine the oocytes with elite sperm. The embryos are cultured for three days in a group dish, then the viable embryos are transferred to fresh medium in individual wells of a culture dish on day 3. On day 5, 25 μl of medium is removed from each well of the dish and transferred to a well of a dish from a kit that is preloaded with reaction mixture comprising dNTPs, Bsu polymerase, 10×TSPY primer master mix as described in example 1, except using SEQ ID NOs: 7-12, and SYBR Green I. The mix is incubated at 37° C. for 90 minutes. Approximately half of the wells turn yellow, meaning the embryos are male. These are discarded. The other half are implanted into surrogate mothers and gestated until birth.

### Example 5

This example illustrates a method of determining the presence of a desired edit using a method of the present teachings.

Primers for LAMP can be designed across the breakpoint for a CRISPR based gene deletion in CD163 and then combined in a 10× master mix with a ratio as described in Example 1. Ribonucleoprotein complexes comprising gRNAs designed to edit the CD163 gene can be introduced to ten porcine zygotes which are then individually cultured to the blastocyst stage. 2 μl of media from each culture are then added to a tube with 12.5 μl of WARMSTART® Colorimetric LAMP 2× Master Mix, 2.5 μl of 10× primer mix, and 8 μl of water. The mixture can be incubated for 40 minutes at 61° C. Three of the ten reaction mixtures might turn yellow. The three blastocysts corresponding to the positive tests can be implanted into surrogate mothers and gestated until birth.

### Example 6

This example illustrates the use of a panel of SNPs used in reactions of the present teachings for use in calculating an EBV.

Primers will be designed for a panel of 96 traits based on the presence of certain alleles in the genetic background and 10× primer mixes prepared as described in Example 1 (but using trait-directed primers). Whole genome amplification is performed on media from several cultured blastocysts using the QIAGEN® REPLI-g® (QIAGEN® Group, Germantown, MD) kit according to manufacturer directions. 2.5 μl of these primer mixes will then individually arrayed onto a 96 well plate—one primer mix/trait per well. A labelled probe that hybridizes to a desired form of the trait and a sink probe that hybridizes to the alternate form of the trait in question will also be added to the well, for a total of 5 μl. Reaction master mixes will be prepared using 5 μl of REPLI-g® amplification product from culture and 10 μl of 2×LAMP master mix comprising polymerase, dNTPs, and water. 15 μl of master mix will be added to each well. Reactions will be incubated at 60-70° C. (depending on oligos for the SNP panel) for 45 minutes, and then the results (presence or absence of fluorescence from the desired form of the trait) will be read using a plate reader. The results for each trait will then be used to calculate an estimated breeding value for each of the blastocysts. The best blastocysts will be implanted into surrogate mothers and gestated until birth.

### Example 7

This example illustrates screening of embryos for a desired trait.

The bovine haplotype HHP carries the trait polled, which means an animal that does not grow horns. It is a common animal husbandry practice to dehorn animals to prevent them from injuring each other or their handlers. The polled trait alleviates this requirement, however the trait has been historically associated with adverse phenotypes, so it is not as prevalent in elite germplasm. Genetics companies are currently developing elite animals that are homozygous for HHP.

One animal that is heterozygous for HHP can be bred with a homozygous wild type animal and will produce HHP homozygous (polled) animals in Mendelian ratios—50% will be heterozygous polled animals, and 50% will be wild type homozygotes. By utilizing IVF to mate two such animals, a breeder can take advantage of the present teachings by creating a plurality of embryos from such a mating. LAMP primers are designed around the locus such that a labelled loop primer binds to the mutation in the polled gene that causes the trait. A 10× primer mix directed to the polled locus is created as described in Example 1. The plurality of embryos from the heterozygous mating are then cultured to the blastocyst stage, and 5 μl of the culture media are added to 12.5 μl of LAMP Master Mix comprising dNTPs, PAR, manganese ions, and Bsu DNA polymerase, 2.5 μl of the 10× primer mix, and 5 μl of water. The mix is incubated at 60° C. for 30 minutes. Positive reactions will turn yellow, indicating which cultures contain embryos that carry the polled mutation. These embryos are implanted into surrogate mothers and gestated until birth.

### Example 8

This example illustrates the detection of a deleterious haplotype in blastocysts.

HH6 is a recessive, embryonic lethal mutation in the SDE2 gene that shows no adverse changes in expression of the SDE2 protein in heterozygous animals. However, this mutation can lead to a 25% reduction in fertility due to embryonic death of HH6 homozygous animals. Because this mutation is newly discovered (2018), the gene is still present in the population due to genetic drift.

A breeder wishing to breed two heterozygous animals carrying the HH6 haplotype can do so without risking an increase in lost pregnancies by obtaining primers directed to the HH6 locus and a HEX labelled probe that binds to the HH6 mutant locus and a Cy3 labelled probe that binds to the wild type locus, mating the two animals via IVF, and then collecting media at the blastocyst stage. 7 μl of media is mixed with 2.5 μl of 10× dNTPs, 8 μl of water, and 12.5 μl of a master mix comprising: dNTPs, and Bst polymerase. The mix will be incubated at 65° C. for 30 minutes under a fluorescent light provided with the kit. Reactions with HH6 mutant alleles will turn green. Blastocysts that test negative (reactions that turn yellow), will be implanted into surrogate mothers and gestated until birth.

### Example 9

This example illustrates use of LAMP to determine the sex of bovine blastocysts in serum-free medium.

1000 bovine zygotes will be cultured in serum-free medium.

300-400 blastocysts will be selected. The following will be mixed together: 12.5 μl of WARMSTART® Colorimetric LAMP 2× Master Mix (New England Biolabs, Ipswich, MA), 2.5 μl of the LAMP primer mix as described in Example 1, 2.5 μl media removed from the blastocyst cultures at day 7, and water to a total of 25 μl. The mix will be incubated at 61° C. for 50 minutes. The tubes with culture from putative male blastocysts will turn yellow, the tubes from cultures with putative female blastocyst will turn pink. For validation purposes, sex determination will be confirmed by real time PCR analysis.

### Example 10

This example illustrates the use of cell free DNA for embryo genotyping and early selection of bovine embryos.

750 oocytes will be matured and mixed with sperm via standard IVF methods and cultured for three days. 96 blastocysts will be placed in separate wells of a 96 well plate and allowed to grow for three days. Medium will be removed and then whole genome amplification will be performed using the REPLI-G® kit according to manufacturer's directions. The resulting amplified DNA will be run on a BovineLD v2.0 Genotyping BeadChip (Illumina) in order to ascertain the genotype of the embryo. Predicted Transmitting Abilities, which are genotype-based value models, will be calculated based on the SNP sequences, which in turn will allow for the calculation of selection indices like NM$. The top animals based on their NM$ will be implanted into surrogate mothers and gestated until birth.

### Example 11

This example illustrates the use of the present teachings for sex determination in porcine embryos.

LAMP primers will be designed against the porcine HSFY gene. In vitro produced embryos will be cultured in individual wells for several days. The following will be mixed together: 12.5 μl of WARMSTART® Florescent LAMP 2× Master Mix (New England Biolabs, Ipswich, MA), 2.5 μl a LAMP primer mix in ratios similar to those described in Example 1, 5 μl of media removed from the cultures, and 7 μl of water. The reactions will be incubated at 61° C. for 30 minutes. Reactions that have a positive signal will be presumptive boars that will be implanted into surrogate mothers and gestated until birth.

### Example 12

This example illustrates the use of fluorescent intercalating dye in LAMP sexing assays.

A 10× primer mix using TSPYN1 primers (SEQ ID NO: 1-6) was created as described in Example 1. 3 μl of media from 5-day blastocyst culture was combined with 12.5 μl of WARMSTART® 2× Master Mix, 0.5 μl of LAMP fluorescent dye (NEB, catalog number B1700S), 2.5 μl of the 10× primer mix, and water to a total volume of 25 μl. The reaction was then incubated for 30 minutes at 60° C. Fluorescence was analyzed on an RT-PCR machine, and presence of the Y chromosome was detected.

### Example 13

This example illustrates the use of cell free DNA for genotyping of porcine embryos.

In vitro produced porcine embryos are cultured in individual wells for several days. Cell free DNA from spent culture media is then subjected to multiple displacement amplification. The amplified DNA is then run using the GGP Porcine LD Array (ILLUMINA®). The resulting SNP genotypes are then used to create an estimated breeding value via BLUP. Animals with the best EBVs are implanted into surrogate mothers and gestated until birth.

### Example 14

This example illustrates the detection of a deleterious haplotype in blastocysts.

HH0 causes Brachyspina syndrome (HH0), which is a congenital inherited lethal defect in Holstein cattle that causes embryonic death, stillbirth and other deformities. (e.g., TY).

A breeder wishing to breed two heterozygous animals carrying the HH0 haplotype can do so without risking an increase in lost pregnancies by obtaining primers directed to the wild type HH0 locus. The following will be mixed together: 12.5 μl of WARMSTART® Colorimetric LAMP 2× Master Mix (New England Biolabs, Ipswich, MA), 2.5 μl of the LAMP primer mix for wild type HH0 in ratios as described in Example 1, and 2.5 μl media removed from the blastocyst cultures at day 7. The mix will be incubated at 61° C. for 55 minutes. Tubes with medium from embryos having a wild type gene will turn yellow. Homozygous HH0 mutants will remain pink.

The medium from embryos that test positive for the wild type gene can further be tested for the mutant allele by repeating the LAMP reaction described supra using primers directed to the mutant allele of HH0. Positive reactions are presumptive heterozygous for the mutation and may be discarded. Embryos that are positive for the wild type allele and negative for the mutant allele may be prioritized for implantation.

### Example 15

This example illustrates the detection of a deleterious haplotype in blastocysts.

HH1 is a nonsense mutation in the APAF1 gene. A breeder wishing to breed two heterozygous animals carrying the HH1 haplotype can do so without risking an increase in lost pregnancies by obtaining two sets of primers: one directed to the wild type APAF1 locus and one directed to the mutant APAF1 locus and performing assays like those described in Example 14.

### Example 16

This example illustrates the detection of a deleterious haplotype in blastocysts.

HH3 is an embryonic lethal phenotype that is caused by an SNP in the SMC2 gene causing a single amino acid substitution. A breeder wishing to breed two heterozygous animals carrying the HH3 haplotype can do so without risking an increase in lost pregnancies by obtaining sets of primers directed to the wild type and mutant SMC2 locus and performing assays like those described in Example 14.

### Example 17

This example illustrates the detection of a deleterious haplotype in blastocysts.

HH4 is an embryonic lethal caused by genetic lesions in the GART gene. A breeder wishing to breed two heterozygous animals carrying the HH4 haplotype can do so without risking an increase in lost pregnancies by obtaining sets of primers directed to the wild type and mutant GART locus and performing assays like those described in Example 14.

### Example 18

This example illustrates the detection of a deleterious haplotype in blastocysts.

HH5 is an embryonic lethal caused by genetic lesions in the TFB1M gene. A breeder wishing to breed two heterozygous animals carrying the HH5 haplotype can do so without risking an increase in lost pregnancies by obtaining sets of primers directed to the wild type and mutant TFB1M locus and performing assays like those described in Example 14.

### Example 19

This example illustrates the detection of a deleterious haplotype in blastocysts.

JH1 is an embryonic lethal mutation in CWC15 found in Jersey cattle. A breeder wishing to breed two heterozygous animals carrying the JH1 haplotype can do so without risking an increase in lost pregnancies by obtaining sets of primers directed to the wild type and mutant CWC15 locus and performing assays like those described in Example 14.

### Example 20

This example illustrates the detection of a deleterious haplotype in blastocysts.

Jersey Neuropathy with Splayed Forelimbs (JNS) calves affected with Jersey Neuropathy with Splayed Forelimbs are unable to stand and exhibit significant extensor rigidity of forelimbs and/or excessive lateral abduction. This condition is caused by a UCLH1 missense variant located at 60,158,901 on the sixth chromosome.

A breeder wishing to breed two heterozygous animals carrying the JNS haplotype can do so without risking JNS calves or carriers by obtaining sets of primers directed to the wild type and mutant UCLH1 locus and performing an assay like that described in Example 8.

### Example 21

This example illustrates a method of determining the presence of a desired edit using a method of the present teachings.

Primers for LAMP can be designed across the breakpoint for a CRISPR based gene deletion in the Prolactin Receptor (PRLR) and then combined in a 10× master mix with a ratio as described in Example 1. Ribonucleoprotein complexes comprising gRNAs designed to edit the PRLR gene can be introduced to ten bovine zygotes which are then individually cultured to the blastocyst stage. 5 μl of media from each culture are then added to a tube with 12.5 μl of WARMSTART® Fluorescent LAMP 2× Master Mix, 2.5 μl of 10× primer mix using ratios similar to those used in Example 1, and 5 μl of water. The mixture can be incubated for 30 minutes at 61° C. Three of the ten reaction mixtures might have fluorescent signal. The three blastocysts corresponding to the positive tests can be implanted into surrogate mothers and gestated until birth.

### Example 22

This example illustrates screening of embryos for a desired trait.

The bovine trait slick is a dominant mutation in PRLR that results in animals with shorter hair. Having shorter hair allows the animal to better tolerate heat and heat stress. This is a dominant mutation.

One animal that is heterozygous for the slick mutation can be bred with a homozygous wild type animal and will produce slick animals in Mendelian ratios—50% will be heterozygous slick animals, and 50% will be wild type homozygotes. By utilizing IVF to mate two such animals, a breeder can take advantage of the present teachings by creating a plurality of embryos from such a mating. LAMP primers are designed around the locus such that a labelled loop primer binds to the mutation in the prolactin receptor gene that causes the trait. A 10× primer mix is created as described in Example 1. The plurality of embryos from the heterozygous mating are then cultured to the blastocyst stage, and 3 μl of the culture media are added to 12.5 μl of LAMP Master Mix comprising dNTPs, PAR, manganese ions, and Bsu DNA polymerase, 2.5 μl of the 10× primer mix, and 7 μl of water. The mix is incubated at 60° C. for 40 minutes. Positive reactions will be read in a fluorescent plate reader, and fluorescence will indicate which cultures contain embryos that carry the slick mutation. These embryos are implanted into surrogate mothers and gestated until birth.

### Example 23

This example illustrates the use of a biofluid for LAMP amplification of a desired trait.

A farmer uses sperm from a polled bull to artificially inseminate several heifers that are heterozygous for the polled trait. Once calves are born, she collects saliva from each calf. Two separate LAMP assays are run on each saliva sample: one with a fluorescent probe specific to the wild type allele and a sink probe specific to the polled allele. The other with a fluorescent probe specific to the polled allele and a sink probe specific to the wild type allele. Calves that are negative for the polled allele are culled.

### Example 24

This example illustrates the sexing of pigs using saliva samples.

10 ropes were hung above mixed sex pig pens. The pigs in the pens chewed on the ropes for enrichment, depositing saliva on them. Saliva samples were collected from the ropes by squeezing the ropes. A porcine testes sample was used a positive control. 400 μl of sample were processed using the QIAAMP® DNA Blood Minikit (QIAGEN® Group, Germantown, MD) according to the manufacturer's directions. Primers for Real Time PCR (RTPCR) were designed as follows:

TABLE 5

| | | Primer/probe | SEQ ID | | |
|---|---|---|---|---|---|
| Gene | Location | Name | NO: | Sequence | Label |
| EIF1AY | Y | pEIF1AY_F | 35 | TTGAGAGCATCCGAGACA | |
| | Chromosome | | | GA | |
| | | pEIF1AY_R | 36 | GCCCAGGGAGTAAATCAC | |
| | | | | AC | |
| | | pEIF1AY_Probe | 37 | TTGTTCCTCCTTTACGCC | VIC*/ |
| | | | | AC | |
| AR | X- | PAR-12_F | 38 | TGCACACCCACTTTCACT | |
| | Chromosome | | | TC | |
| | | PAR-12_R | 39 | AGGGGACCAGCAAGTATG | |
| | | | | AA | |
| | | PAR-12_Probe | 40 | AACAGTGCCTGCTCAAAG | FAM |
| | | | | AAA | |
| Glucagon | Autosome | pGCG_F | 41 | GTCCTGGCTCTGCACTTT | |
| | | | | TC | |
| | | pGCG_R | 42 | TCAATTGGCTGTGATGGG | |
| | | | | TA | |
| | | pGCG_Probe | 43 | CAATTCCCTTTCCAACAG | CY5 |
| | | | | ACA | |

2 μl of 5 ng/μl DNA was mixed with 10 μl of SYBR Green Master Mix, 1 μl of each 5 pmol/μl forward (F) and reverse (R) primer, and water to a total volume of 20 μl. The mixture was divided in half (10 ul) to make two technical replicates and then heated at 95° C. for 2 minutes, then subjected to forty cycles of 15 seconds 95° C. and 1 minute of 60′ C. Results are shown in Table 6.

TABLE 6

| Sample | Porcine genomic targets Presence | | |
|---|---|---|---|
| ID | EIF1AY | AR | Glucagon |
| Male 1 | Yes | Yes | Yes |
| Male 2 | Yes | Yes | Yes |
| Male 3 | Yes | Yes | Yes |
| Male 4 | Yes | Yes | Yes |
| Female 1 | No | Yes | Yes |
| Female 2 | No | Yes | Yes |
| Female 3 | No | Yes | Yes |

These results illustrate that extracellular DNA can be isolated from porcine saliva and used to determine the presence of differential markers.

### Example 25

This example illustrates validation of a quantitative PCR assay for determining the ratio of male DNA to female DNA.

This experiment utilizes control male DNA isolated from pig testes tissue and control female DNA isolated from cumulus cell tissue. For this multiplexed reaction, the primers and probe for each reaction were combined into a Primer Mix. 20 μl each of 100 μM forward and reverse primers were combined with 5 μl of 100 μM probe with 155 μl of water. For each PCR reaction 10 μl of TaqPath™ ProAmp™ Master Mix (Thermo Fisher Scientific, Waltham, MA), 1 μl of an EIFlAY primer mix (SEQ ID NOs: 32-34), 1 μl of an AR primer mix (SEQ ID NOs: 35-37), 2 μl of DNA template (10 ng/μl), and 6 μl of nuclease free water. PCR was performed with 2 minutes of activation at 500 C, 2 minutes of denaturation at 95° C., and then 40 cycles of 1 second at 95° C. denaturation followed by 20 seconds at 60° C. Different ratios of control DNA were tested.

TABLE 7

| | X (AR) | Y (EIF1AY) | Difference in Cycles |
|---|---|---|---|
| Sample | Detected? | Detected? | to Detection? |
| Male only | Yes | Yes | No |
| Female only | Yes | No | N/A |
| Male: Female (1:1) | Yes | Yes | No |
| Male: Female (1:19) | Yes | Yes | Yes, Y slower |

This qPCR assay was able to detect differences in Y chromosome presence at higher female:male ratios.

### Example 26

This example illustrates the differentiation of male and female pigs based on saliva samples.

Saliva samples were collected from female pigs and boars and then DNA was purified as described in Example 24. These DNA samples were then used to perform qPCR as described in Example 25. Results are shown in Table 8.

TABLE 8

| Sample | X (AR) Detected? | Y (EIF1AY) Detected? |
|---|---|---|
| Female 1 Aliquot 1 | Yes | No |
| Female 2 Aliquot 1 | Yes | No |
| Boar 1-03/26 | Yes | Yes |
| Boar 2-03/26 | Yes | Yes |

This assay was able to differentiate between female and male pigs based on cell free DNA samples obtained from saliva.

### Example 27

This example illustrates comparison of a colorimetric LAMP sex determination assay of the instant teachings relative to a standard qPCR assay.

A LAMP assay was performed on 100 blastocysts according to the procedure described in Example 1 using the spent medium. In parallel, DNA was isolated from each blastocyst and the sex determined using qPCR. The two results were compared to assess the accuracy, specificity, and sensitivity of the assay. Accuracy was assessed as the total number of embryos where the LAMP assay correctly assessed the sex of the embryo (as compared to qPCR). The sensitivity of the assay was measured using the number of male embryos correctly diagnosed (times the assay detected amplification when it should have been present). The specificity was measured using the number of female embryos accurately diagnosed (times the assay did not detect amplification when there should have been none). The results are shown in Table 9.

TABLE 9

| Embryos | qPCR | LAMP | Assay Index (%) | |
|---|---|---|---|---|
| Male | 58 | 37 | 64 | Sensitivity |
| Female | 42 | 41 | 98 | Specificity |
| Total | 100 | 78 | 78 | Accuracy |

These results illustrate that this LAMP assay is as specific as qPCR but not as accurate or sensitive.

### Example 28

This example illustrates an HSFY directed LAMP assay.

This assay sought to amplify the HSFY gene. A 10× primer mix was made as shown in Table 10.

TABLE 10

| | SEQ | 10× | 1× |
|---|---|---|---|
| | ID | Concentration | Concentration |
| Primer | NO: | (Stock) | (Final) |
| HSFY_FIPe | 46 | 16 μM | 1.6 μM |
| HSFY_BIPe | 47 | 16 μM | 1.6 μM |
| HSFY_F3 | 44 | 2 μM | 0.2 μM |
| HSFY_B3 | 45 | 2 μM | 0.2 μM |
| HSFY_LF | 48 | 4 μM | 0.4 μM |
| HSFY_LB | 49 | 4 μM | 0.4 μM |

Then a master mix was prepared (per reaction ingredients are shown in Table 11).

TABLE 11

| Reagents (2×) | 1 Rxn (in ul) |
|---|---|
| Water | 1.9 ul |
| 10× Reaction Buffer | 2.0 ul |
| (Varigen) | |
| 10 mM dNTP | 2.0 ul |
| HSFY Primer Mix (10×) | 2.0 ul |
| 100 mM MgSO4 | 1.2 ul |
| 50× SYTO82 (0.5 mM) | 0.4 ul |
| LAMP Enzyme (Bolt | 0.5 ul |
| Polymerase, Varigen) | |

10 μl of master mix was then dispensed into PCR tubes. 5-10 μl of spent media from single embryo culture was then added to each well. Nuclease free water was added to bring the total reaction volume to 20 μl if needed. The reaction was spun down to the bottom of the tube and then cycled in a qPCR machine for 45 minutes at 62° C. and data were analyzed in Quantistudio 5 (Thermo Fisher) to determine male and female embryos.

### Example 29

This example illustrates a comparison of an HSFY LAMP sex determination assay at 8 days of culture to qPCR results to determine the quality of the assay.

Media was removed from 61 day 8 single bovine embryo cultures and used in the assay described in Example 29. DNA was then isolated from each blastocyst and used for qPCR. The results were compared and the results are shown in Table 12.

TABLE 12

| Embryos | qPCR | LAMP | Assay Index (%) | |
|---|---|---|---|---|
| Male | 32 | 22 | 69 | Sensitivity |
| Female | 29 | 29 | 100 | Specificity |
| Total | 61 | 51 | 84 | Accuracy |

These data illustrate that this assay is highly specific and accurate.

### Example 30

This example illustrates a comparison of an HSFY LAMP sex determination assay at 7 days of culture to qPCR results to determine the quality of the assay.

Media was removed from 205 Day 7 bovine embryo cultures and used in the assay described in Example 29, using 6 μl of media for each assay. (This allowed for four replicates.) DNA was isolated from each blastocyst for sex determination using qPCR. The results are shown in Table 13.

TABLE 13

| Embryos | qPCR | LAMP | Assay Index (%) | |
|---|---|---|---|---|
| Male | 127 | 77 | 60.62992 | Sensitivity |
| Female | 78 | 76 | 97.4359 | Specificity |
| Total | 205 | 154 | 75.12195 | Accuracy |

These data illustrate that the assay is highly specific and very accurate using 6 μl of 7 day culture.

### Example 31

This example illustrates assay quality of an HSFY LAMP sex determination assay using 3 μl of culture.

12 μl of media from 60 embryo cultures was split into four aliquots of 3 μl each. These aliquots were then used in a LAMP reaction similar to Example 28, except that more water was required to bring the reaction up to 20 μl. DNA was isolated from the blastocysts and used for qPCR. The results from each assay were compared. These results are shown in Table 14.

TABLE 14

| Embryo | qPCR | LAMP | Assay Index (%) | |
|---|---|---|---|---|
| Male | 39 | 22 | 56 | Sensitivity |
| Female | 21 | 20 | 95 | Specificity |
| Total | 60 | 42 | 70 | Accuracy |

These data illustrate that using smaller amounts of media for multiple replicates results in only modest changes in assay quality.

### Example 32

This example illustrates the use of all 12 μl of an embryo culture in a HSFY LAMP sex determination assay of the present teachings.

Media was harvested from 57 embryos and then all 12 μl of media was used in an assay using the protocol of Example 28, except that water was omitted to bring the volume to 20 μl. DNA was then isolated from each blastocyst. The comparative data are shown in Table 15.

TABLE 15

| Embryos | qPCR | LAMP | Assay Index (%) | |
|---|---|---|---|---|
| Male | 37 | 20 | 54 | Sensitivity |
| Female | 20 | 19 | 95 | Specificity |
| Total | 57 | 40 | 70 | Accuracy |

These data illustrate that larger amounts of media can be used without detrimental effects on assay quality.

### Example 33

This example illustrates the quality of in a HSFY LAMP sex determination assay of the present teachings using Day 8 embryos.

Media was harvested from 107 embryos and all the media about 12 μl was used in an HSFY LAMP assay as described in Example 28. DNA was harvested from the embryos and used in a qPCR assay. The comparative data are shown in Table 16.

TABLE 16

| Embryos | qPCR | LAMP | Assay Index (%) | |
|---|---|---|---|---|
| Male | 60 | 46 | 77 | Sensitivity |
| Female | 47 | 47 | 100 | Specificity |
| Total | 107 | 93 | 87 | Accuracy |

These data illustrate that using media from day 8 embryos increases the specificity and accuracy of the assay.

### Example 34

This example illustrates titration of different amounts of media from embryo cultures.

Embryos were grown in 20 μl, 15 μl, or 10 μl of culture media. 5 μl of media from individual embryo cultures were harvested. 2× master mixes were prepared as described in Example 28. DNA was isolated from embryos and used for qPCR. The data are shown in Table 17-Table 19.

TABLE 17

| Embryo (20 μl media) | qPCR | LAMP | Assay Index (%) | |
|---|---|---|---|---|
| Male | 41 | 19 | 46 | Sensitivity |
| Female | 32 | 29 | 91 | Specificity |
| Total | 73 | 48 | 66 | Accuracy |

TABLE 18

| Embryos (15 μl media) | qPCR | LAMP | Assay Index %) | |
|---|---|---|---|---|
| Male | 41 | 17 | 41 | Sensitivity |
| Female | 40 | 38 | 95 | Specificity |
| Total | 81 | 55 | 68 | Accuracy |

TABLE 19

| Embryo (10 μl Media) | qPCR | LAMP | Assay Index (%) | |
|---|---|---|---|---|
| Male | 32 | 14 | 44 | Sensitivity |
| Female | 31 | 31 | 100 | Specificity |
| Total | 63 | 45 | 71 | Accuracy |

These data illustrate that changing the culture volume to 10 μl leads to an increase in assay accuracy and specificity.

### Example 35

This example illustrates an HSFY assay of the present teachings using SYBR Green and VIC fluorescent dyes.

To test the feasibility of the HSFY assay using multiple dyes, 500 μg of sperm DNA and 500 μg of cumulus cell DNA were mixed with 4 μl of bOEC2 culture media for positive controls. A LAMP reaction was prepared as described in Table 11. The samples were placed in a qPCR machine programmed as depicted in Table 20.

TABLE 20

| Cycle condition | | Cycle no |
|---|---|---|
| 15 Sec | Ramp to | 60× |
| | 62° C. at | |
| | 1°/sec | |
| 15 sec | 62° C. | |

Data capture on second step

Signal was detected at approximately 30-35 cycles for male DNA and not detected in female DNA. These data demonstrate that the assay performs as expected using DNA of known genotype.

### Example 36

This example illustrates amplification of three DNA types using whole genome amplification.

4 μl of culture media was removed from a 7 day blastocyst embryo culture. Whole genome culture was performed by using the QIAGEN® REPLI-g® whole genome culture kit according to manufacturer directions. The amplified cell-free DNA was then used as a template for qPCR. Primers were directed to 1.75 (V00125.1, autosomal), TSPY (NM_001244608.1, Y chromosome), and 12S (MN714218, mitochondrial). 1.75 product appeared at about 15 cycles, TSPY product at about 20 cycles, and 12S product at about 25 cycles. These robust products illustrate that all three types of DNA are present in cell free DNA.

### Example 37

This example illustrates results of an HSFY assay using culture from a day 8 media culture.

8 μl of media was removed from 107 bovine blastocyst cultures and used in the assay described in Example 29. DNA was then isolated from each blastocyst and used for qPCR. The results were compared and the results are shown in Table 21.

TABLE 21

| | | | Assay Index | |
|---|---|---|---|---|
| Embryos | qPCR | LAMP | (%) | |
| Male | 60 | 46 | 77 | Sensitivity |
| Female | 47 | 47 | 100 | Specificity |
| Total | 107 | 93 | 87 | Accuracy |

These data illustrate consistent assay specificity and accuracy between day 7 and day 8 cultures (see Example 34).

All publications cited herein are hereby incorporated by reference, each in their entirety.

1. A method of making an animal or a cell line having a trait from an in vitro produced embryo comprising:
combining male and female gametes in vitro to form an embryo or producing a parthenogenetic embryo;
culturing the embryo or parthenogenetic embryo for a period of time sufficient for cell-free DNA to accumulate in medium;
adding the medium to an amplification reaction mix comprising primers targeted to a trait-related locus and at least one turbidimetric, colorimetric, or fluorescent indicator;
amplifying the cell-free DNA;
determining that the primers amplify the trait-related locus or determining that the primers do not amplify the trait-related locus based on a signal from the at least one turbidimetric, colorimetric, or fluorescent indicator;
selecting the embryo or parthenogenetic embryo; and
implanting the selected embryo into a surrogate mother or using cells from the selected embryo or selected parthenogenetic embryo to create a cell line.

2. The method according to claim 1, wherein the selecting the embryo or parthenogenetic embryo comprises selecting the embryo or parthenogenetic embryo if the trait-related locus is amplified by the primers.

3. The method according to claim 1, wherein the selecting the embryo or parthenogenetic embryo comprises selecting the embryo or parthenogenetic embryo if the trait-related locus is not amplified by the primers.

4. The method according to claim 1, wherein the amplifying the cell-free DNA is by loop-mediated isothermal amplification (LAMP).

5. The method according to claim 1, wherein the trait is a health trait, a reproductive trait, a disease resistance trait, an anatomical trait, a desired gene edit, or sex of the embryo.

6. The method according to claim 1, wherein the trait is sex of the embryo or the parthenogenetic embryo and the locus is on the Y chromosome.

7. The method according to claim 1, wherein the trait-related locus is TSPY or HSFY.

8. The method according to claim 7, wherein the primers comprise a set of oligonucleotides consisting of: SEQ ID NO: 1-6, SEQ ID NO: 7-12, SEQ ID NO: 3-6, and 13-14, SEQ ID NO: 9-10, 15-17, and 34, SEQ ID NO: 7-10, 17, and 34, SEQ ID NO: 9-12 and 15-16, SEQ ID NO: 26-31, SEQ ID NO: 28-33, SEQ ID NO: 5-6 and 13-14, SEQ ID NO: 15-17 and 34, SEQ ID NO: 26-27 and 30-31, SEQ ID NO: 1-2 and 5-6, SEQ ID NO: 7-8, 17, and 34, SEQ ID NO: 30-33, or SEQ ID NO: 44-49.

9. The method according to claim 8, wherein a female embryo is desired and the embryo or parthenogenetic embryo is selected if the at least one turbidimetric, colorimetric, or fluorescent indicator indicates that the primers do not amplify TSPY or HSFY.

10. The method according to claim 8, wherein a male embryo is desired and the embryo or parthenogenetic embryo is selected if the at least one turbidimetric, colorimetric or fluorescent indicator indicates that the primers do amplify TSPY or HSFY.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. An embryo or parthenogenetic embryo produced according to the method of claim 1.

25. The method according to claim 1, further comprising determining a value based on a genotype-based value model and selecting the embryo or parthenogenetic embryo based on the value.

26. (canceled)

27. The method according to claim 1, wherein the period of time is 3-9 days.

28. The method according to claim 1, wherein the period of time is about 7 days.

29. The method according to claim 1, further comprising determining a value based on a genotype-based value model and selecting the embryo or parthenogenetic embryo based on the value.

30. The method or kit according to claim 1, wherein the trait-related locus is a target of gene editing for a desired edit, and the primers are directed to the desired edit.

31. The method according to claim 1, wherein the embryo or parthenogenetic embryo is a non-human mammalian embryo or a non-human parthenogenetic mammalian embryo.

32. The method according to claim 1, wherein the embryo or parthenogenetic embryo is a bovine embryo, a bovine parthenogenetic embryo, a porcine embryo, or a porcine parthenogenetic embryo.

33. (canceled)

34. The method according to claim 1, wherein the embryo or parthenogenetic embryo is a non-human embryo or a non-human parthenogenetic embryo.


SEQUENCE LISTING (66 sequences)

SEQ ID NO: 1 - DNA, 46 bp, Bos taurus
  source 1..46: mol_type = other DNA
tgacgcttcctcctctgatggtttttgagcaaaaccgcagggctta

SEQ ID NO: 2 - DNA, 44 bp, Bos taurus
  source 1..44: mol_type = other DNA
cgccatcatccagggcatccttttatcaggacggagacttgagg

SEQ ID NO: 3 - DNA, 20 bp, Bos taurus
  source 1..20: mol_type = other DNA
cctgcaggtagaactgagct

SEQ ID NO: 4 - DNA, 20 bp, Bos taurus
  source 1..20: mol_type = other DNA
agtcttgatcttggtcgctg

SEQ ID NO: 5 - DNA, 20 bp, Bos taurus
  source 1..20: mol_type = other DNA
tcttgcgcatgaactgaacg

SEQ ID NO: 6 - DNA, 18 bp, Bos taurus
  source 1..18: mol_type = other DNA
ctggcttctgggccaaag

SEQ ID NO: 7 - DNA, 44 bp, Bos taurus
  source 1..44: mol_type = other DNA
tgacgcttcctcctctgatggtttttagctctgagcgtgagcaa

SEQ ID NO: 8 - DNA, 43 bp, Bos taurus
  source 1..43: mol_type = other DNA
cgccatcatccagggcatccttttgctgatcaggacggagact

SEQ ID NO: 9 - DNA, 19 bp, Bos taurus
  source 1..19: mol_type = other DNA
cgccctgcaggtagaactg

SEQ ID NO: 10 - DNA, 20 bp, Bos taurus
  source 1..20: mol_type = other DNA
agtccatcatgtagccgaga

SEQ ID NO: 11 - DNA, 19 bp, Bos taurus
  source 1..19: mol_type = other DNA
tgaactgaacgtaagccct

SEQ ID NO: 12 - DNA, 22 bp, Bos taurus
  source 1..22: mol_type = other DNA
gggccaaagctattatgagcca

SEQ ID NO: 13 - DNA, 42 bp, Bos taurus
  source 1..42: mol_type = other DNA
tgacgcttcctcctctgatggtgagcaaaaccgcagggctta

SEQ ID NO: 14 - DNA, 40 bp, Bos taurus
  source 1..40: mol_type = other DNA
cgccatcatccagggcatccatcaggacggagacttgagg

SEQ ID NO: 15 - DNA, 40 bp, Bos taurus
  source 1..40: mol_type = other DNA
tgacgcttcctcctctgatggtagctctgagcgtgagcaa

SEQ ID NO: 16 - DNA, 39 bp, Bos taurus
  source 1..39: mol_type = other DNA
cgccatcatccagggcatccgctgatcaggacggagact

SEQ ID NO: 17 - DNA, 21 bp, Bos taurus
  source 1..21: mol_type = other DNA
catgaactgaacgtaagccct

SEQ ID NO: 18 - DNA, 40 bp, Bos taurus
  source 1..40: mol_type = other DNA
tccacccagtgagctccaagagggaagcaaagaaccccgc

SEQ ID NO: 19 - DNA, 39 bp, Bos taurus
  source 1..39: mol_type = other DNA
gtcaagcctcctgaggcgttagagatgagggccttcgtc

SEQ ID NO: 20 - DNA, 18 bp, Bos taurus
  source 1..18: mol_type = other DNA
cggggagacaggcattca

SEQ ID NO: 21 - DNA, 20 bp, Bos taurus
  source 1..20: mol_type = other DNA
gagaacaaccccgagattgg

SEQ ID NO: 22 - DNA, 20 bp, Bos taurus
  source 1..20: mol_type = other DNA
cgtcacaattcgagagcaga

SEQ ID NO: 23 - DNA, 19 bp, Bos taurus
  source 1..19: mol_type = other DNA
tggagagaggtcgcgagat

SEQ ID NO: 24 - DNA, 44 bp, Bos taurus
  source 1..44: mol_type = other DNA
tccacccagtgagctccaagagttttggaagcaaagaaccccgc

SEQ ID NO: 25 - DNA, 43 bp, Bos taurus
  source 1..43: mol_type = other DNA
gtcaagcctcctgaggcgttttttagagatgagggccttcgtc

SEQ ID NO: 26 - DNA, 40 bp, Bos taurus
  source 1..40: mol_type = other DNA
tgttcctgctcctcatccgccaccagctgcttgcggaaga

SEQ ID NO: 27 - DNA, 39 bp, Bos taurus
  source 1..39: mol_type = other DNA
agctagaggagaagacggtggatcctggcgctcacaagg

SEQ ID NO: 28 - DNA, 16 bp, Bos taurus
  source 1..16: mol_type = other DNA
ggatcgggcgggagtt

SEQ ID NO: 29 - DNA, 16 bp, Bos taurus
  source 1..16: mol_type = other DNA
tgcctgcacggcatcc

SEQ ID NO: 30 - DNA, 20 bp, Bos taurus
  source 1..20: mol_type = other DNA
cctccacctcctccatgatg

SEQ ID NO: 31 - DNA, 17 bp, Bos taurus
  source 1..17: mol_type = other DNA
agcagggccaggaaagg

SEQ ID NO: 32 - DNA, 44 bp, Bos taurus
  source 1..44: mol_type = other DNA
tgttcctgctcctcatccgccattttccagctgcttgcggaaga

SEQ ID NO: 33 - DNA, 42 bp, Bos taurus
  source 1..42: mol_type = other DNA
agctagaggagaagacggtggattttcctggcgctcacaagg

SEQ ID NO: 34 - DNA, 21 bp, Bos taurus
  source 1..21: mol_type = other DNA
ggccaaagctattatgagcca

SEQ ID NO: 35 - DNA, 20 bp, Sus scrofa
  source 1..20: mol_type = other DNA
ttgagagcatccgagacaga

SEQ ID NO: 36 - DNA, 20 bp, Sus scrofa
  source 1..20: mol_type = other DNA
gcccagggagtaaatcacac

SEQ ID NO: 37 - DNA, 20 bp, Sus scrofa
  source 1..20: mol_type = other DNA
ttgttcctcctttacgccac

SEQ ID NO: 38 - DNA, 20 bp, Sus scrofa
  source 1..20: mol_type = other DNA
tgcacacccactttcacttc

SEQ ID NO: 39 - DNA, 20 bp, Sus scrofa
  source 1..20: mol_type = other DNA
aggggaccagcaagtatgaa

SEQ ID NO: 40 - DNA, 21 bp, Sus scrofa
  source 1..21: mol_type = other DNA
aacagtgcctgctcaaagaaa

SEQ ID NO: 41 - DNA, 20 bp, Sus scrofa
  source 1..20: mol_type = other DNA
gtcctggctctgcacttttc

SEQ ID NO: 42 - DNA, 20 bp, Sus scrofa
  source 1..20: mol_type = other DNA
tcaattggctgtgatgggta

SEQ ID NO: 43 - DNA, 21 bp, Sus scrofa
  source 1..21: mol_type = other DNA
caattccctttccaacagaca

SEQ ID NO: 44 - DNA, 24 bp, Bos taurus
  source 1..24: mol_type = other DNA
tgtctcaaatatcttcattagtgc

SEQ ID NO: 45 - DNA, 20 bp, Bos taurus
  source 1..20: mol_type = other DNA
ggtggttaggtaagtcacat

SEQ ID NO: 46 - DNA, 49 bp, Bos taurus
  source 1..49: mol_type = other DNA
gcgctctctctactaatttctggaaagataacaagaagcatgttaaagc

SEQ ID NO: 47 - DNA, 46 bp, Bos taurus
  source 1..46: mol_type = other DNA
ttcagcctccacaagtttacctgcatttgtattagtgacaatctgg

SEQ ID NO: 48 - DNA, 24 bp, Bos taurus
  source 1..24: mol_type = other DNA
cagagttacgatcatcttcgttga

SEQ ID NO: 49 - DNA, 21 bp, Bos taurus
  source 1..21: mol_type = other DNA
gtgcctttcatacaaaagcct

SEQ ID NO: 50 - DNA, 1200 bp, Bos taurus
  source 1..1200: mol_type = other DNA
atgtcgcgtcccttcgcctctgccccagctcggggtcaccgccaaggccaagaggaacgtgagaggcgctcagaggaaggcgggagcgtcccgggaccccgcaccttccaagttgtgagcccagttgtcacgccaggccaagaggccacccgcttcagggtggaggcggtggaggagggcgaggccctggtggacggagacgtggcggggatcgggcgggagttccagctgcttgcggaagacatcatggaggaggtggaggttgtggcggatgaggagcaggaacagcggccctcccaggagctagaggagaagacggtggaggagcagggccaggaaaggcccggaggcccttgtgagcgccaggagctggatgccgtgcaggcactggccgccctgcaggtagaactgagctctgagcgtgagcaaaaccgcagggcttacgttcagttcatgcgcaagaaccatcagaggaggaagcgtcacttggctcggaggagcgccatcatccagggcatccctggcttctgggccaaagctattatgagccaccctcaagtctccgtcctgatcagcgaccaagatcaagactttctcggctacatgatggacttgaaggtgcaggtgcggagccatccgccgtcccgctgcaagctgatcttttcctttcgggacaacccctacttcttgaactcggtgatcattaaggagtattaccttgacatcactgggtacagggcacgtcgatccactccagtccactggttctgggactttgaacggggagcccccagccgcaggctggacaccaggagccttaactttctcaactggctgtcaggccacaacggcccagaatcgaacaggattgctgagatcatcagcgaagacgtgtgggacgatcccctgaagtactacctcagggaggaaggttcgtccatgagagacaactgacagaaacacatgtggtgatggggtctggagggcggacgcagagatgaaggcgacgggagaggaggtgacgagcgaccagaagaggaggcacgaggcgtccccaggctgacggaggttggtcaccgactctccgcatgaccgtatccgggatgggaatgaggaaaagatggccagtgatacatcctgcggaccgtggctcgagaaatccgcaattagaccaaatccaaaacacgctcgccacttcta

SEQ ID NO: 51 - DNA, 123 bp, Sus scrofa
  source 1..123: mol_type = other DNA
gaactcgggtggggcccccatgcccgcrcattccccwctggaggcmctgaaagcscttcagttggagctagagcctgtgaataarcaagcagccggggccttctckcggctgaagctcaggac

SEQ ID NO: 52 - DNA, 928 bp, Bos taurus
  source 1..928: mol_type = other DNA
gttgatgggtttgggctgactgccaggacgtattgaggggaggtattgggggcggagaaataaatatttcactgtatatattgcactaagtcagtctgtggtaagaacaacttatgaatagcaccataatttttagaacgcttacaccgcatattacttcctccccttttaaacagtgcagtcgtatgcttctgctatgttcagagtattgaacgacgatgtttacagtccagctgtggtacagcaacaaactactctcgcttttaggaaagactcttccttgtgcacagacagtcatagcgcaaatgatcagtgtgaaaggggagaacatgttagggagagcagccaggaccacgtcaagcgacccatgaacgccttcattgtgtggtctcgtgaacgaagacgaaaggtggctctagagaatcccaaaatgaaaaactcagacatcagcaagcagctgggatatgagtggaaaaggcttacagatgctgaaaagcgcccattctttgaggaggcacagagactactagccatacaccgagacaaatacccgggctataaatatcgacctcgtcggagagccaagaggccacagaaatcgcttcctgcagactcttcaatactatgcaacccgatgcatgtagagacattgcaccccttcacatacagggatggttgtgccaagaccacatactcacaaatggaaagccaattaagccggtcacagtccgtgatcataaccaattcactcctgcaaaaggagcatcacagcagctggacaagcctgggccacaataaggtaacattggctacacggatttcggcggactttccctgtaacaaaagcttagagcctggactttcttgtgcttattttcaatattgacttccttactctcgctaacaaaggcgctctttatctcaattt

SEQ ID NO: 53 - DNA, 943 bp, Sus scrofa
  source 1..943: mol_type = other DNA
cccagcggtttccaggggaggtactgggggcggagaaattggtatttcactacaaagattagagttactcaaatctctggtggaaataacctttaaatagtgaagacaactttccaaacgcttacgcttttgatttcgcttcctcccccttttcaaatggtgcagtcatatgcttctgctatgttcagagtattgaaagcggacgattacagcccagcggcacagcagcaaaatattctcgccttggggaaaggctcctcactatttccgacggacaatcatagctcaaacgatggacgtgaaactagaggaagtggtagagagagtggccaggatcgtgtcaagcgacccatgaacgctttcattgtgtggtctcgtgatcaaaggagaaaagtggctctagagaaccctcaaatgcaaaactcagagatcagcaagtggctgggatgcaagtggaaaatgcttacagaagccgaaaagcgcccattcttcgaggaggcacagaggctacaggcggtgcaccgagataaatacccgggctataaataccgacctcgtcgcaagggagagagggcacagaatttgcttccggcagaggcggcagtactatgcagccaagtgcgcgtagaggagaggatgtatcccttcacatacacagtcgccaaggccaagtgctcaggaacagaaagccagttaagtcactcacagcccatgaacataaccagctcacttctgcaacaggaggatcgctgcaactggacaggcctgtgccacagtagggtaacatccaccgggcagatccgcgccgacttgccttttcaccgtggtttacagccgggactttctcacatttattttccatattgatttcctttactgtcgcgaacagagggcctattcatctcagttttactgttatttcacctgtgacttagtttcagat

SEQ ID NO: 54 - DNA, 2179 bp, Bos taurus
  source 1..2179: mol_type = other DNA
tcattcaagagagtttgtttccggtagaccaacctgtgagccggttcccgcgggagtcgtttcaaatactgatacgctagacaagccatttgagacagagttcgcgcctttcttcaggcatgagtcatgaggcggtgaaaaacgtgcaggaggtggatcagcagttggctgatctaaacctgaactctgataataatcagagtggaggaggaagtacagctagcaaagggcgctatataccacctcacttaaggaatagggaagcatctaaaggattttatggtaaaggcagttcagggtggagttgtagtaaagaaaaggatgcctacagcagttttggatctcgagattcaagaggaagacccagttacttcagtgatcgaggaagtggatcaaggggaaggtttgatgatcgtgggcggaatgagtgtgaaggttttggcagtcgtggtgacagaactggctttggcagatttgagcgtagtggacacagtcgttggtgtgacaagtcagatgatgatgactggtcaaaaccgcttccaccaagtgaacgcttggagcaggagctcttttctggaggaaacactgggattaactttgagaaatatgatgatataccagtagaggtaaccggcaataactgtcctccgcacattgaaagtttcagcgatgttgcaatgggagaaattattatgggaaacattgagcttactcgttacacacgtcctactccagtgcaaaaacatgccattcccattattaaagaaaaaagggacctgatggcctgtgcccagacagggtctgggaaaactgcagcatttcttttgcccatcttgagtcagatttatacagatggtccaggcgaggctttgaaagctgtgaaggaaagtggaaggtatggacgccgtaaacagtatccaatctccttggttttagccccaacaagagagttggctgtacagatctatgaggaagccaggaaattttcataccggtctcgagttcttccttgtgtggtatacggtggtgctgatattggtcaacaaattcgagacttagaacgtggatgtcacttgttggtggccactccaggacgcctggtggatatgatggaaaggggaaagattgggttagatttctgcaaatacttggtgttggatgaagctgataggatgctggatatgggatttgaacctcagatacgtcgtataattgaacaagacactatgccaccaaagggtgttcgccataccatgatgtttagtgctacttttcctaaggaaattcagatgcttgctcgtgatttcttggatgaatatatctttctggccgtaggtagagttggctctacctctgagaacatcacacaaaaagtagtttgggtggaagaggcagacaaacggtcatttctgcttgatctcttaaatgcaacagggacagattcactgactttagtttttgtggagacaaaaaagggggctgattctttggaggatttcttataccatgaaggatatgcttgtacaagtatccatggagaccgatcacagagagatagagaggaagcacttcatcagttccgctcaggaaaaagcccaattctagtggctactgctgtggcagcaagaggactagacatttcaaatgtgaaacatgtcatcaattttgatttgccaggtgatattgaggaatatgtacatcggattggccgtacaggacgtgtaggaaaccttggtcttgccacctcatttttcaatgaaagaaatataaatatctcaaaagatttgttggatcttcttgttgaagccaaacaagaagtgccatcttggttagaaaacatggcttatgaacaccactataagagtagcaatcgtggccgttctaggagattcagtggaggctttggtgcccgtgattatcgacaaagtagcagttctggcagtttcagctatagtagtggtcgtgcaggcagcagccgcagtggtggaggtgctcatggcagcagcagaggatttggtggaggtggctatggaggcttctacagtagtgaaggatatggcggaaattacagctctcagggggttgactggtggggcaactgaatctgctttccagccaagtcacttaaaaaaaagctacaatggaaaccacaaaaaaaaaaaaaaaaaaaaaaaaaaaa

SEQ ID NO: 55 - DNA, 2006 bp, Sus scrofa
  source 1..2006: mol_type = other DNA
tcttcaggcatgagtcatgtggtggtggaaaatgcgcttggtctggaccagcagtttgctggtctagacctgaactcctctgataatcagagtggaggaggaagtacagcaagcaaaggtcgatatatacctccccacttaaggaacagagaagcatctaaaggatattatgataaagacagttcaggatggagttgtagtaaagataaggacgcctatagcagttttgggtctcgagattcaagaggaaaacccagttacttcagtgatcgaggaagtggaacaaggggaaggtttgacgatcgtggacggaatgattatgatggtatgggcagtcgtggtgacagaactggctttggcaaatttgaacgcagtggacacagtcgttggtgtgacaagtcagatgaagatgactggtcaaaaccacttccaccaagtgaacgcttggagcaggaactgttttctggaggaaacactggaattaactttgagaaatatgatgatataccagtagaggcaactggcaataactgtcctcgacatattgaaagtttcagcgatgttgagatgggagaaattatcatggggaacattgagcttactcgttatactcgtcctactccagtgcagaaacatgccattcctattatcaaagaaaaaagagatttgatggcttgtgcccaaacagggtctggaaaaactgcagcatttcttttgccaattttgagtcagatttatacagatggtccaggcgaggctttgaaggctgtgaaggagaatggaaggtatggacgccgtaaacaatacccaatttccttggttttagctccaacaagagaattggctgtacagatctatgaggaagccagaaaattttcataccggtctcgagttcgtccttgtgtggtatatggtggtgctgatattggtcagcagattcgagacttagaacgtggatgtcacttgttagttgccactccaggacgcctggtggatatgatggaaagaggaaagattgggttagatttttgcaaatacttagtgttggatgaagctgatagaatgttggatatggggtttgaacctcaaatacgtcgtatagttgaacaagatactatgccaccaaagggtgttcgccataccatgatgtttagtgctacttttcccaaggaaattcagatgcttgctcgtgatttcttggatgaatatatctttctggctgtaggtagagttggctctacctctgagaacatcacacagaaagtagtttgggtagaagagtcagacaaaaggtcatttctgcttgacctcctaaatgcaacagggaaggattcactgactttagtttttgtggagaccaaaaagggtgccgattctttggaggacttcttataccatgaaggatatgcttgtaccagtattcatggagaccgatcacagagagatagagaggaagcccttcaccagttccgctcaggaaaaagcccaattctagtggctactgctgtggcagcaagaggactagacatttcaaatgtgaaacatgttatcaattttgatttgccaagtgatattgaggaatatgtacatcggattggccgtacaggacgtgtaggaaaccttggccttgccacctcatttttcaatgaaagaaatataaatatcacaaaggatttattggatcttcttgttgaagctaaacaggaagtaccatcttggttagaaaatatggcatatgaacatcattataagggtagcagtcgtgggcgttctaaaagattcagtggaggatttggtgcccgagactatcgacaaagtagtagttctggaagttcgacctttagtagtagtcgtgcaggcagcagtcgcagtggtggaggtggtcacagcagcagcagaggatttagtggaggtggctatggaagcttctacaatagtgatggatatggaggaaattataactcccaaggggttgactggtggggcaactgaatctcctttgcagc

SEQ ID NO: 56 - DNA, 450 bp, Bos taurus
  source 1..450: mol_type = other DNA
gagcctgccgctacagctatgcccaagaataaaggtaaaggaggtagaaacagacgcaagggtaagaaggagaatgaatatgaaaaaagagacttagtctttaaagaagacggacaagaatatgctcaggtaaccaaaatgttgggaaatgcacgattggaagcaatgtgttttgatggagtgacaaggatatgtcatatccgaggaagcttgagaaaaaaggtttggataaatacttcggatattatattgattggtcttcgagactatcaggacaacaaagctgatgtaattttaaagtacaatgctgatgaagctagaagtctgaaggcttatggagagcttccggaatatgccgaacttagtgaaacagatacctttgatgctgaagatgatgatgaaatccagtttaaggaccctgcaggtgaggatgaagacatggaggtgtaa

SEQ ID NO: 57 - DNA, 1053 bp, Sus scrofa
  source 1..1053: mol_type = other DNA
ggaacattagaggagtttgcgttagacgtttgtaattgcgcccaacatttcagaggagtatttacctgcgccacaaacacctgaacgcatgttaaactcagagcggccgcggttaccggcatgcctaagaataaaggtaaaggaggtaaaaacaggcgcaggggtaagaatgagaatgaatctgaaaaaagagaattggtgtttaaagaggatggacaagaatatgctcaggtcatcaaaatgttgggaaatggaagattggaagcaatgtgttttgatggtgtaaagaggttatgtcatatcagaggaaaattgagaaaaaaggtttggataaatacctcagatattatattggttggtcttcgtgattatcaggataacaaagctgatgtaattttaaagtacaatgcagatgaagctagaagtctgaaggcttatggggagcttccagaacatgcaaagattaatgaaactgatacatttggtcctggagatgatgatgaaatccagtttgatgacattggagatgatgatgaagacattgatgatatctaaattggtcttacaagagtttctgaggattattctacagtttgaatttcggtcatcacctgttaagattaaaacttcattcaagatgaatgtagtcttaagacacactgatttattttaagtggtctaaagtatttattatatttctttaaacaactgatgaagctgaattattttaagtaaaatgttaacactgttcttttaatgtaatggaatgtaggggtaaaagaccataatatctacattaaaacacttggaagggagaaagaagaaaagagatgcctttaccactgtaattagtttacttccagttcctggatgttttaaataaattgctgtatcattatggactgtgataagccccagttcctgctatctctttacagagtctttctctatattctgatttgcctaaatgttctcttgagatttttgataaaatttaaggaaagcagaaatctgcatttatattaacaaacggggctgcttttgtatgtgtgt

SEQ ID NO: 58 - DNA, 1851 bp, Bos taurus
  source 1..1851: mol_type = other DNA
gagagtttctctctgttggccagccaagcaatttttgaagtagtggtcagtaaaatcagacaaatgagtaaaggatctgaagaaagatttctctgagatggcacatataccttcagaaattcaagatgggcctcctaaggatgaatcaactgattcagaaacctccattagatctttgtatgattatacacttactagggactcagttttgagatctatgattgaagaatatgcttttcaggctttgtctgaggatcttgtgataaaaaggccatgctacaaatattctgtatctgaaacagatgaggtgactaattttctttcactcacctttccacaaaaactttggaatatagttgaaagtgatcagtttgaatctatttggtgggatgagagaggcacatgtatagtgatccacgaagaactctttaagaaagaagtcttggaaagaaaggcacctttcagaatatttgaaaccaagagtatgaagagtttaattcgacagcttaacctttatggatttagtaaaaagcgacaaacttttcaaagatctgcttcactacctgtctttctggaagaagaaaacaacatctctcttttgagtaaggtattccaacattttcacttattggcaatacgtaagatgaaatcatgtgacctagaaagcgcatttacatatgtagctaaaaccgaatttaaacttgagctaacaaatcgttaaaaagttttattttttgaagttgagaacagctggatatgttaaaatattggatgtttgacaaactttcctagcactgtgaaaccagatataagtcctgaagcaacttaaagtggtatttactgtatatataaaatatatttttcattgaggatcatattctttctaacatgttacctgttaaactactaatggaggtgttttatttgatgctatttagttggtttcattaaaggaatacatttttataacttagctttgcttgtcagttttaccttggtttataaagaaactaaataatgtaatcctttgattttagttacagacctactataatccaaatttcaaaagaggtcatcctcaacttttattaagagtgcaaagaagagaaagaagagttgggattaataatgtgtctcaaatatcttcattagtgcaagataacaagaagcatgttaaagcaagtgtcaacgaagatgatcgtaactctgaatttattccagaaattagtagagagagcgcattttcagcctccacaagtttacctgtgcctttcatacaaaagcctcataccatccagattgtcactaatacaaatgccctatctccatgtgacttacctaaccacccatcaccaatatcggttagacaaacagaacagattttggtaaatcaacctgctgttttaaaaaagttgagcatttttaattggcattcacatagcagctacactcaagtaaatggccccattgaggactttgctactacaactacatctacttctcagaaccacattgtatccccattacagagcacttattctggactgatggtggagccttctaaatttccagtcaggcatagcgatatgtcaggccatgacaatccttttcctaacctgcaagagacaggcaactcgtggttctcagtgccaacaagcacttacacatctgcttcctctctttcaagtcaactcatcaacaatcaccattatatggaaaccatgctaattaaaactgacctaccaaatacgtgtcagattatggagcctaaggaagattgaacgttactttgggagatgtcaacaaatacctaccattcctgtatttgaacaataaatttacatgttcatc

SEQ ID NO: 59 - DNA, 1425 bp, Sus scrofa
  source 1..1425: mol_type = other DNA
gaaagcattttaaaactgcagagttcaaaagccatgcaggtgagaaaagtagctgaagagaaatttatctgagatggcacatgctccttcagaaattcaaggtgtgtctcctaaagatgaatcaactggttcagaatcctccattacatctccatggtgtgataactcaatcactggggacttggacttgaggtctcagattgaagaaaaagctttccaggctttgtctgagggctccctgatgaaagagccatgccacacattgtgtgcctctgaaccagatggagataatgattttcctcctctgccctttcccagaaaactttgggaaattgttgaaagtgatcgatttaagtccatttggtgggatgaaaatggaacctcgatcgtgattaatgaagaacgcttcaagaaagaagttctggagagaaaggctcccttcagaatttttgaaactgagagtatgaaaagcttagttcgacagcttaacctttatggatttactaaattgcgaaggaattttcaaagacctgcctcactagccaacattctggcagaagataatgaagtctctgcttggaacaagctgcagttctaccataatccaaattttaaacgaggctgtccccatctgttggtgaggatgaaaagaagagtcagggtgaaaaatgcttctccagtatcggcttctctacttcaaggttgcagcaagaagcgctctagaacagggggtgatgtggataaccataattctgatgtggctgcagaaactcgtggagaagatgcattttcagcctctacaaacctaaatgtgcctttaataagatggccttctatgagccagagaatttctaatacaactaccccaactagaagtggtctttcttctccatcatcaccatcagtcaggccaccaggacaaattgtcatggctcaacatgctatttttagtcagttgaccacgtttcacatgcactcacaaagcagctactctccagcaagtggccacatcgtgaatttcattacaactaccacttccatttctcagtaccgccttatttctaacttcccaagtggttattttggactgagggtggagccttctcctttgccaaccagataccagaatatatcaccaactgagtgccctgtgtctaacccgcaaccagcagcccatccatggttcccaatgccaatggccactgacacatctgctgcctcgctttcaaggccaaatcctcagtcctcttccgtatctgaacatcaccctaattaccactgacctatcaaaggactataggttatgcaggagagtgaagatgaaaatttatgctggcaaatgtcaccaaattcctgcaattctgttatttcagcaataaacttgtatgttcatcttcaaaaaaaaaaaaaaaaaaaaaaaaaaaa

SEQ ID NO: 60 - DNA, 427 bp, Sus scrofa
  source 1..427: mol_type = other DNA
aagttttttaggtggaaaaatagtattgtgggttttgtttaaaagaaaagtcattatgctagggtttttttgtttttgtttttgttttcgttttttgtctttttagggccacacctgtagcatatggaggttcccaggctaggggttgaattggagctacagctgctggcctacgccacagccacagcaatgctggatctgagcaccatttgcaacctacgccacagctcacagcaactttggacccttaagccactgaatgaggccagggatggaacctgcatcctcatggatactggtaagattaatttccactgagccgcacgggaattccatgtgtttttgaagtttagttgtgtacaggtttttttactctaaataaaaataagttaactttttttcagattaaccaagttactcatgatca

SEQ ID NO: 61 - DNA, 7782 bp, Bos taurus
  source 1..7782: mol_type = other DNA
gcaaaaatcttttctgttttgagtatgacagccactactcgtggctctccagtaggagggaatgacagccagggccaggctcctgatggacagtctgagacccccttccagaatcagacctcatcacctgattcttccaatgagaattccccagcaactcctccagatgaacaaggtcaaggtgatgctccacctcaacatgaagatgaggaacctgcatttcctcatgctgacctggcaaagttggatgacatgatcaaccggcctcgttgggtggttccagttttgccaaaaggggaattagaagtgcttttagaagctgctattgatcttagtaaaaaaggtcttgatgttaaaagtgaggcatgtcaacgttttttccgagatgggctaacaatatctttcactaaaattcttatggatgaagcagtgagtggctggaagtttgaaattcatagatgcattattaacaatactcatcgcctagtggaactttgtgtggccaagttgtcccaagactggtttccacttctagaacttctcgccatggccttaaatcctcattgcaaatttcatgtctacaatggtacacgtccttgtgaatcagtttccacaagtgctcagtcacctgaagatgaattattcgctcattcctcagatcctcgatcaccaaaaggttggctagtggatctcatcaataaatttggcatattaaatgggttccagattttgcatgatcgttttattaatggatcagcattaaatgttcaaataattgcagctcttattaaaccatttggacaatgctatgagtttctcaatcaacatacagtgagaaagtactttattccaattatagaaatagttccacaatttttagaaaatttaactgatgaagagctgaaaaaagaagcaaagaatgaaactaaaaatgatgccctttcaatgattattaagtctctgaagaatttagcttcaaggattccagaccaagaagaaactgtaaaaaatttagaaatttttaggttgaaaatgatactcagattgttgcaaatttcttcttttaatgggaagatgaatgccctgaatgaaattaacaaggtgatttctagtgtatcatattatactcatcggcatggtaatcctgaagaggaagaatggctgacagctgagagaatggcagaatggatacagcagaacaatatcttatccatagtcttgcgagatagtcttcatcaacctcagtatgtagaaaagctagagaagattcttcgttttgtgattaaagaaaaagctcttacgttacaagatcttgataatatctgggcagcacaggcagggaaacacgaagccattgtaaagaatgtacatgaccttctggcaaagttggcttgggatttttctgctgaacaacttgatcatctttttgattgctttaaggcaagttggacaaatgcaagtaaaaagcaacgtgaaaagctgctagagctgatacgacgtcttgcagaagatgataaagatggtgtgatggcacacaaagtgttgaaccttctttggaatctggctcatagtgatgatgtgcctgtagacatcatggacctagctctcagtgcccacataaaaatactagattatagttgttcccaggatcgagacacacaaaagatccagtggatagatcgctttattgaagaacttcgcacaaatgacaaatgggtaattcctgcactgaaacaaataagagaaatttgtagtttgtttggggaagcacctcagaatttaagtcaatctcagcgaagtccccatgtattttatcgtcatgatttaatcagtcagcttcagcacaatcatgccttagttactttggtagcagaaaaccttgcaacttacatggatagcacacgactatatgctagagaccatgaagactatgacccacaaactgtgaggcttggaagtagatacagtcatgttcaagaagttcaagaacggcttaacttccttaggtttttattaaaggatggccagctgtggctttgtgcgcctcaggcaaaacaaatatggaaatgcttagcagagaacgcagtttatgtttgtgatcgtgaagcctgttttaagtggtattccaagttaatgggggatgaaccagacttagatcctgatattaataaggaattctttgaaagtaatgtgcttcaacttgatcctttcctgttaactgaaaatggaatgaaatgttttgaacgattcttcaaagctgttaattgtcgagaagggaaactagtaacaaaaagaagagcctatatgatggatgatttggaactaataggattagactacctttggagagtcgtaattcagagtaatgatgatattgccagcagagctatagatctccttaaagaaatatatacaaatcttggccccagattacaggtcaatcaggtggttatccatgaggactttattcagtcttgctttgatcgtttgaaagcttcatatgatacattgtgtgttttggatggtgacaaagacagcattaactgtacaaaacaggaagctgttcgaatggttcgagtattgactgttttaagggaatacataaatgaatgtgatagtgattatcatgaggaaagaacaattctacctatgtcaagagcattccgtggtaaacatttgtctctcatagttcggtttccaaaccatggcagacaggttgatgatttagatatatggtctcatacaaatgatactgttggttcagtacgacgatgtattctcaatcgtattaaggcaaatgtagcccatacaaaaattgaactctttgtgggtggtgaattaatagattctgaagatgacagaaagctaattggacagctgaacttaaaagacaaatcaataattacggccaaacttacacaagtaagttgcaatatgccttccagtcctgatagttcttctgattcctcaactggatctcctgggaactattgtaagcgttacagtgatggccctaaacaagaagtggaaagttgtttgcctggggtgataatgtcacaacatcccaggtacatttctttcctttggcaagttgctgacttaggtagcaatctgaatataccacctcttagagatggagccagaatacttatgaaacttatgccaatggatagcacaacagtagaaaaactaagagctgtttgtttggaccaggcaaaacttggagaaaataaccttggaccttctcttgattcacttttctttggtccttctgcttcccgagtcctgtatctaacagaggttgtttatgccttgttaatgcctgctgatatgcctctggcagatgattcttgtgactttcagtttcacttcttgaaaagtggtggcttatcccttgtactgagtatgctaaccaaaaataacttcctgccaaatacagatatggaaacacgaaggggtgcatacttcagtggtcttaaaatagccaaactcttgttaactgcagtaggctatggccatgttagagctgtagcagaagcttgtcagccagttgtagatggtgcagatcccataacaccgattaaccaagttactcatgatcaagcagtggtgctacaaaatgctcttcagagcattcctaacccattatctgagtgtattcttagaaatgtatcaatacggcttgctcaacaaatatctgaagaggcttcaagatatatgcctgatatttgtgttattagagctatacagaaaatcacctgggcatcaggatgtgggacattagagctagtatttagccctaatgaagaaatcactaaaatttacgagacgaccagcagtccaagccatgaactagatgtggaagatgaacaggtttgctgtgaagcattggaagtgatgactttatgttttgctctagttccaacagccctggatacacttaataaagaaaaagcttggcagacatttatcattgacttattattgcattgtcccagcaaaattgttcgtcagttggcacaggaacagttttttttaatgtgcaccagatgttgcatgggacacagacctctgcttttcctcattactttgctctttaccattttggggagcacagcaagagagagaggtaaacattcaggtgactatttcagtcttcttcggcaccttcttaattatgctttcaacaataatattaatgtgcccaatgctgaagttcttctcaacaatgaaattgactggctcaaaagaattagggataatgttaaaagtagaggtgaaacaggtattgaagagcctatcttggaaggccaccttggggtaacaaaagagttactggcctttcaaacacctgagaaaaagtatcatattggttgtgaaagaggaggcgctaatcttgttaaagaattaatagatgatttcatctttcctgcctccaaagtttacctgcagtgtatgagaaatggagaactaccagctgaacaggctatcccagtctgtagttcaccagctaccatcaatgctgggtttgaactacttgtagcattagctgttggctgtgtaaagaatctcaaacagatagtagactgtttgactgaaatgtattacataggaacagcaataactacttgtgaagcacttacggagtgggaatatctaccacctgttggaccccgcccaccaaaaggatttgtgggactcaaaaatgctggtgctacttgttatatgaattctgtgatccagcagttatacatgattccttccatcaggaatagtattcttgcaattgaaggcacaggtagtgatattgatgatgatatgtttggtgatgagaagcaggacagtgaaagtaatgttgacccccgagatgatgtatttggataccctcatcaatttgaagacaaaccagcattaaataaaacagaagataggaaagagtacaatattggtgttctgagacaccttcaggttatctttggtcatttagctgcttcccggctacaatactatgtccccagaggattttggaaacagttcagactttggggtgagcctgttaatcttcgtgaacaacatgatgccttagagttttttaattctttggtggatagtttagatgaagctttaaaagctttaggacatccagctatgctaagtcaagtcctgggaggttcctttgctgatcagaagatttgccagggctgcccacatagatatgagtgtgaagaatcttttacaactctgaatgtagatattagaaatcaccaaaaccttctggattctttggaacagtatatcaaaggcgatttattggaaggtgcaaatgcttatcgttgtgaaaaatgtgataaaaaggttgatacagtaaaacggttgctaattaaaaaattgcctcctgttcttgctattcaattgaaacgttttgactatgactgggaaagagaatgtgcaattaaattcaatgattattttgaatttccacgagagctggacatggaaccttacacagtactaggtgttgcaaagctggaaggagataatgtaaacccagagagtcagttgatggaacagaatgaacggtctggaagtgatacaactggaaacacgaaatacagacttgtgggggtgcttgtacacagtggtcaagcaagtggtggacattattactcctacatcattcagaggaatggcaaagatggtgacagaaatcgctggtataaatttgatgatggagatgtaacagagtgtaaaatggatgatgatgaagaaatgaaaaatcagtgttttggtggagagtacatgggagaagtatttgatcatatgatgaaacgcatgtcatacagaaggcagaaaagatggtggaatgcttatatattgttttatgaacgaatggacacaatagaccaagaagatgaaatgataagatacatatcagagctaactatcacaaaaccccatcagattatgtcaccagccattgagagaagtgtgcgaaaacaaaatgtgcaattcatgcataaccgaatgcagtacagtttagaatattttcagtttgtgaaaaaattgcttacatgtaatggtatttacttaagccctgctccaggacaagatcacttactgcctgaagctgaagaaattactatgattagcattcagcttgcagctaggttcctctttaccactggatttcacaccaagaaaatagtccgtggccctgccagtgattggtatgatgcattgtgcattctactccgtcacagcaagaacgtacgtttttggtttgctcataatgtgcttttcagtgtttcaaatcgtttctctgaataccttctggagtgccctagtgctgaagtgaggggtgtatttgcaaaacttatagtatttgttgcacatttttctttgcaagatgggccttgttcttcatctttcacatctccaggaccttctagtcaggcatgtgacaatttaagtttgagtgatcatctactaagagcagtgttaaatctcctgagaagggaagtgtctgaacatggtcgtcatttacagcaatatttcaatctgtttgtcatgtatgccaatttaggtatggcagaaaagacacagcttctgaaattgagtgtacctgctacctttatgcttgtgtctttagatgaaggaccaggtcctccaatcaaatatcagtatgctgaattaggcaagttgtactcagtagtgtctcaactgatccgctgttgcaatgtctcatcgagaatgcagtcttcaatcaatggtaatccccctcttcccaatcctttcggtgaccctaatatatcgcagcctataatgccaattcagcagaatgtggcagacattttatttgtgagaacaagctatgtgaagaaaattattgaagactgcagtaattcagaggaaaccatcaaattgcttcgtttttgctgctgggagaatcctcagttctcgtctacagtcctcagtgaactactctggcaggttgcatattcatacacctatgaacttcggccatatttggatctacttctgcagattttactgatagaggattcctggcaggctcacagaattcataatgcacttaaaggaatcccagatgatcgagatggattgtttgatactatccagcgctctaagaatcactatcaaaaaagagcataccagtgtataaaatgtatggtggctctctttagcaattgtcctgttgcttaccaaatattacagggcaatggagatcttaaaagaaagtggacctgggcagtagaatggcttggagatgaacttgaaagaagaccatacactggcaatccacaatacacttataataattggtctcctccagttcagagcaatgaaacatcaaatggttatttcttagagagatcacatagtgctaggatgacactggcaaaagcttgtgaactctgtccagaggaggagccagatgaccaagaagccccagatgagcatgaatcatctccaccagaagatgccccattatatcctcattcatctggatctcactatcaacagaataatcacatacatggacagccatatacaggaccagcagcacatcacttgaacaaccatcaaaaaactgcccagcagcaacaagagaactgtgaaggcaatgaagaagtatcctcttctgaagtgaaggatcagtgaaatgtccataattacctggttctttaagaccaggctattaggccttacagtccaaacttttatatacttggttagtatttaacaaaatagagaaattgt

SEQ ID NO: 62 - DNA, 2406 bp, Bos taurus
  source 1..2406: mol_type = other DNA
atggatgaagatgaatttgaattacagccacaggagccaaactcatgttttgatggaataggaactgatgctacacacatggatggtgatcaaattgttgtggaagtacaagaaactgtttttgtttcagatgttgtggattcagatataactgtgcataattttgttcctgatgatccagactcagttgtaatccaagatgttattgagaatgttgttattgaggatgttcagtgctcagatatcttagaagaagcagatgtgtctgaaaatgtcatcattcctgagcaaatgctttcctcagatgtaacagaagaagtgtctttagcacattgcacagtaccagatgacgtcttagcttctgatattacttcagcttcaatgtctatgccagaacatgtcttgaccagtgagtctgtacatgtgtctgatgttggacatgttgaacacattgttcatggtagtgtagtagaggcagaaatcgtcactgatcctctgacagccgacgtagtgtcagaagaagtattggtagcagattgtgcctcagaagcagtcatagatgccaacggaatccctgtggaccagcaggatgatgacaaaggcaactgtgaggactaccttatgatttccttggatgatgatggcaaaatggaacacgactgttcctctggtatgaccatggacgcagagtcggaaatcgatccttgtaaggtggatggcacttgccctgaagtcatcaaggtgtatatttttaaagctgaccctggagaggatgacttaggtgggactgtcgacattgtggagagtgagcctgagaatgatcatggagttgaactccttgatcagaataacagtattcgtatgccaagggaaaagatggtttatatgactgtcaatgactctcaacaagaagatgaagatttaaatgttgctgaaattgcagatgaagtttatatggaagtgattgtaggagaggaagatgctgctgttgctgcagcagctgctaccactgttcatgaacaggaaatggatgacagtgaaatcaaaaccttcatgccaatagcatgggcagcagcttacggtaataattctgatggaattgaaaatcggagtggcactgcaagtgctctcttgcacatagatgagtctgctggacttggcagactgactaaacataaaccaaagaaaaggagaagacctgattccaggcagtaccaaacagcaataattattggtcctgatggacatcccttgactgtctatccttgtatgatttgtgggaaaaaatttaagtcgagaggttttttgaaaaggcacatgaaaaaccatcctgaacaccttaccaaaaagaagtaccgctgtactgattgtgattacactaccaataagaagataagtttacacaatcacctggagagccacaagcttaccagcaagtcagagaaggccatcgaatgtgatgactgtgggaagcatttctcccatgctggggctttgttcactcacaaaatggtgcataaggaaaaaggagccagcaaaatgcataaatgtaaattctgtgagtatgagacagctgaacaagggttattaaatcgccaccttttggcagtccacagcaagaactttccccatatatgtgtagagtgtggtaaaggttttcgtcacccatcagagctcaaaaagcacatgcgaatccatactggagagaaaccgtaccaatgccagtactgcgaatataggtctgcagactcttctaatttgaagacgcatgtgaaaactaagcatagtaaagaaatgtctttcaagtgtgacatttgtcttctgactttctccgataccaaagaggttcagcaacatgctcttatccaccaagaaagcaaaacacaccagtgcgtgcattgtgaccacaagagttcaaactctagtgatttgaaacgtcacataatttcagttcacacaaaggactacccccataagtgtgacatgtgtgataaaggctttcacaggccatcagaactcaagaagcatgtggctgcccacaagggtaaaaaaatgcaccagtgtagacattgtgactttaagattgcagatccatttgttctaagtcgccatattctctcagttcacacgaaagatcttccgtttaggtgtaagagatgtaaaaagggatttaggcaacagaatgagcttaaaaagcatatgaagacacacagtggcaggaaagtgtatcagtgtgagtactgtgaatatagcactacagatgcctcaggctttaaacggcatgttatttccattcatacaaaagactatcctcaccgttgtgagtactgcaagaaagggttccgaagaccttcagaaaagaaccagcatataacacgacaccataaagaagttggtctgccctaa

SEQ ID NO: 63 - DNA, 987 bp, Sus scrofa
  source 1..987: mol_type = other DNA
gtaagagtgattcaatcaaactcttcaacttgattacttttcaggggtttagactgagaatatttgagtttttaatgaaggtaacttttatttatttttggccatgcccacaacaagttcctaagcagaatcagaccacagcagctgctgcattgacaacaccagatctttaagctactgcaccacaagggaattcctgaagacaacctttaaaacagactttcaatgtattaataaaatgttgagtatttattaaggtgaaacaggtggaaatgaagttttcatgtaaattcttggagctctcatcttccttgctagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggagaggaagatgctgctgttgcagccgcagcagctgctgctgtgcatgaacagcaaatggatgacagtgaaatcaaaactttcatgccaatagcatgggcagcagcttatggtaggtcgtgttgcagcttgagggattgcatggttcttgaactttattattgaaaaacagtttctgtggtcttaaattaaaaaatgtaaagtactcatatagtgatttaatcaaaagagaggaagatgatatagtctaccttttgccttttaataggaattttctccatgcttatattacataagacatatgtagtacatagcactgaagtgtatctggctcatgacctttaagtgacaacgagagagtttatggggaagagtgctcctagattgcaaagctagacatttcctgtagttttatatataaatatgcccaataaaagcccaatacctagataggaaatttttgtcattcatgagtatcatatcttttaattgttgtacttaataaaaaatccctagttctttatagtttataatactgtataattttgttttttaatacacattgttag

SEQ ID NO: 64 - DNA, 1381 bp, Bos taurus
  source 1..1381: mol_type = other DNA
atggtgtctgtttcgttttctcagccacaaaaaatacagagccttcttgaagaaggaaaaacgaaagaaacgtcgccgggaactggctagactgagagactcaggactgtcacaaaaagaagaggagaatcctgctgttattgaaaaacagttaaaagaagagaagctgttggagttagagaggcaaaacttacatgatgaatggttattaagggagcagaaggcacaagaggaattcagaataaagaaagaaaaggaagagacagctaggaaacaggaggaagaacaagagacaaacttaaaggaaaaatgggaagaacatcagagaagagagagagaagagaaggagcagaaacaacaggggaaaagagaagaagaggaacctgtgcagaagatgctgaaacaggctgaaaatgagttggaaagtggtaccatatggcataacccagaaccacccatggaaataagaataatggagaaagatcgagctaattgtccattctacagtaaaacaggagcttgcagatttggagataggtgttcacgtaaacacaatttcccaacatcaagtcccacactgcttattaaaagcatgtttacaacgtttgggatggagcagtgtggaagggatgactacgaccctgacgcaaacttagagtacagtgaagaagaaatctaccagcagttcctggacttttatgatgacgtgctccctgagtttaagaacgtggggaaagtgatccagtttaaggtcagttgcaacctggaacctcatctgaggggcaatgtatatgttcagtatcaatcggaagaagaatgccaggcagccctttctctgtttaatggacgatggtatgcaggacgacagcttcagtgtgaattctgcccagtgacccaatggaatatggcaatttgtgtttatttgaaatacagcagtgtcccagagggaaatactgcaactttcttcacgtgttcagaaatccaaacaatgaattttgcgaaggaaatgaagacatctacttacctccaatttggactggctccacctttgggaagagctcagtgaggagggtgaggctggaccaccatgacgactactataacctgcaaaggaggaggaggagccctgtgtgggcccactcctacaagaggaaagacgaggctgacaggaagaggaggagtagtcatgagggcgagaaatctcaaaaacatgggtctagaagccggggaaggcacagttctcaaagcagcggaagaaaaagggaccacagcagcaggaacagcagccagagctcctccatgagccgtggtgggaggaggttggggagcagagactgaacttttcagagccgcaaatccaaataaacttcctttatcactt

SEQ ID NO: 65 - DNA, 3386 bp, Bos taurus
  source 1..3386: mol_type = other DNA
atatttttgatccatgtatagagtctggtgccttctggactcagagaatggagggagtgaggggcacccccaagggacagctgggggctcttttggacattttatgttggtcagcatttctgagtgagattggagatctataagcaaagtggtgcctaccagctctcctgtagagtttaacaggagcactggggccactatggggagacaaggcaaaagtagggtctggagaaggaagtttaaaatgatcatgtcacttaactgagtttgagatgtttccttatgtctaagggtgttaatagttatctcaatgtttcagctgatgattaaatcaggtactaattttgtcttttggaagaaatataatcatcaccacttatctgcagagaataagttccaagacccccagtggacatctgtaaccacacatagtatcaaaccctatatacacaatggtttttctatacaaacatacctgtgatgcagtttattttataaaagaacagtaagagattggcaacttcctaataataacaggaaagtgaaagtgaagttgctcagtcgtgtccaactctttgcaacctgatggactgcagcttactatgctcctctgtgatgggatttcccaggcaagaattctggattgggttgccatttccttctccagggatggaacccaggtctcctgaatagtagcagacactttaccatctgagccaccagggaaggtgtaataataacatagaacaattataacaacatatttttataacagttttgtgaatgtagtctctatgtaaaatatattactgggtctcacgaattccgtgagaccttttttttcaccctaactatgcactatgctctgtggccataccttttgcagtttgcagtttgacagcaaaactaacccagatttcttttctctgcttcacattcaccacagatattagcaacctcagcatacttttttttctttttatatcaaattgagaactttgatcttttcacttaaaggtagcacttaaaggattctctctggcatgtctaaataaccaacatcactccacttacggtttggggccattattgagtaaaataagggtgacttggtcacaagctctgtgacatctcaacagtcattgtggtaaccgagtgacaaatgggtgagatgtgcaggactaagttcgattcacatttctgggcaggaggaagcaggatggtgtgagattccaatcctactcctcagaatgacatgcaatttaaaacttatgagttatgtatttctggagttttccatttaatattttcagtgaacaactgagggttaactgaaaccacagaaagtgacacctgagatcagaggggatgaccttgttctgtggttgcctttaggtggaaaaaacactggaaggggccacttgaagccaaaagtgattgtaaaatggcccatctcttcctatgtgaggaagcacaagcatgaccacaacccgagacaaagaccgcagggtccaaacccagagatgaggataatgatgatgctgaggtcatctttgttggagtgcagcatgtcaatgaggatgctgaaactctctttgtcagagtgatttcaagttcaaagccagtcatttctaacattttgaacagagtgaccagggatgcatcttcaagaagaaagaagggccacgtgagtccagatccctcctgcacattgcaacctgcaaatctcaggaccccagcatcagaaccagtggctgtatcattagcttctcagtctgaatggggaggaaaaaacagtcctgttattttcgagccttcatctaaacctgattataaaacgagctctctggaagctgtgcttcacaattcagaattgctctctctgcggcctcattgtctatctggagctagtctctcaatagcaggcaaggatgaaagtcctcttaattcaaagcaatgtcccacttcagatgtaaaatgtaacagtgggaatcccaaaagacccaaactcagcaatggaatcccagggggacctgcctccaccatggcccctgtaggtatttctcctacaaaggacacgattatgaccttggaaggtctcctgacctcaccgagccatgttcaccgcggggcatcattttcacagactcacgcaaatgaccaggcatgcttcagtcttatggatccagacagaacatgtagttggaggggctggagaaaacggactttttgactcttgcaagtcagagcaagactgttgatcccatgaaaggaaatctgatcatgttacttgatgacttttactatggacagcatacaagagatgggcagtcagaacagaagactcacacagtctttaaatgcctcagttgcttgagagttctaaaaaatgtcaagtttatgacccatgtgaggaaccatctggacctggagaggcagaagggtgacagctgggaaatccacactacctgccagcactgccaccgccagtttcccaccccgttccagctgcagtgtcacattgaaagtgtccatgcttcccgggagccctccacggtctgttatatctgtgaattgtccttcaaaacagatcaggttctcttatagcacatgaaggacagtcacaaacctggcaaaatgccctatgtgtgccgtgtttgcaattacagactgtcagccttggccaatgtagaggtacgttttagaatatggcatggaaacatgaacaatttgctttgtctgttttgcttcgaaattttcaaactggtgacatccttcataaatcacacttggaggcactgaaataagagggtctttccatattccaagtgctggctacagtgtttgaaaatgaaggaaaagacagagcaccaaacaaagaatcatcagtctttcaaaaagccagagcaattgtgagggttgcctcctgaaacagaggtcatgatccaaacttcagttcagctgggactgagagagacagcatccatcattgtgaggaacactgactcccaagtgtcacccagaaaaactacaaagaagattgactacaaacacaaaatattccagacgtgttgcagcaagggatctggctaaaaattaagttctacccacctccagaaattcaaaaaagccctattgaggttcagccagctgtacatagagaccctgcctggcttttgttgttcagtttctcagtcgtgtctgactccctgtgaccctatgaacttcagtatgccaggcttccctgtccatcaccatttcccgaagcttgctcaaactcgttccgttgggttgctgatgccatccaaccatcgtgtcctctgtcacaccttctgctcctgccttcaatagttcccagtatcaggtttgtttgtttgtttgtttgt

SEQ ID NO: 66 - DNA, 1961 bp, Bos taurus
  source 1..1961: mol_type = other DNA
gagttgaagcggcgtcccaggcctgaggaggcattgttccctcgggcctgcctcctcagacttaacccagggactgtttaggtgcaggtgagctgccggcagttctggcatcgccgcccacggggaccggtggcagtttatttaaaggaagcactgggacttgtggtcacaccctccaagaacaaaaaagtatttaaaatatggaaccaccatggatgtcatgtgaagaagaacaagagccagaaccacagaaaagcgagggagaaaccaaactagtaaatgatgaagatgctgaactcatcttggttggagtggaacatgtacatgaggatgatgatgtgatctttgttgggatgacctcaaattcaaaaccagtcatttcaaacatactgaacagagttaccccaggttcttgttcaaggagaaataggtatggtcacttcaggaaagataatgctcagaaattacagcctgttagtcatgtgactcctacatcagaagcaaagactgtcttgccactttctgtctctgaatcaagatcaacagatagtcctattattattgagcctttgtccaaagcggattctaaaaatatttcaccacaaataatgcctaatagcttttcagagttatgttctcctttgattaccttcacaagttcattgcagcatccagtagaaacagcagtttctgcaggagatatggataaaagtcctcgtgtatcaaagcgactttccacttctgaaacaaatagcacaagtcccaaaaggcctacactcagtgatggagttataggagaacattctttagctttgtccctttcaggtacttttcatacagtgactactcagcaaagcacaccagacagtgttcataactcattaagccatgttcagagtggagaaccttgtccaacagcttttccaaaggacaatgttcattgcaagcctgtaagtcctgtagggggaaatgtattgacaaaaactgactttccaagtgtatcaagtcaaaataagtttgctgatcccacagaaggcaatctgattgtgttacttcgtgacttctactatggacagcatataggagatgggcagccagaaccaaaaactcacacagcctttaaatgccacagctgcttgaaggttctaaaaaatgttgagtttatgaatcacatgaagcaccatttggaacttgagaggctgagaggtgacagctggaaataccacaccacctgccagcactgccaacgccagtttcctacgcccttccagctgcagtgtcatattgaaagtgtccacacttcccaggagccctgcacagtctgtaaaatctgtgaattgtcctttgaggcagatcagattctcttacagcacatgaaagataatcataagcctggtgaaatgccctatgtatgccaggtttgcagttacagatcatcattctttgcagatgtggatgcacatttcagagcataccatggtaacactaagaatttactttgccccttttgtctccaaatttttaaaactgaagcactatacatatgtcattataaacagcactgggcaaagcgttttcaccagtgttccaaatgtcggctacaatttttaacttgtaaagagaaaagggagcacaagacccagtgtcatcaaatgtttaagaagcctaagcagctagaaggattacctcctgaaacaaaagttgttattcaggtatcactggaaccccttcaaccaggattggtggaagtagcatccattactgtgaacgcatctgattctgaaccatcaccccccaaatctaaaagtaggaggtcaaaaaaacccggttaagtctactttaagtaagtctgaagcaagtatttcaaagttataaactccattaaaaacaaaaaattcaactatagcattattcaataataaatacagagaaaccaaaaaaaaaaaaaaaaaaaaaaaaaa